(19) United States
(12) Patent Application Publication
(10) Pub. No.: US 2015/0087556 A1
(43) Pub. Date: Mar. 26, 2015

(54) COMPOSITIONS AND METHODS FOR MAKING cDNA LIBRARIES FROM SMALL RNAs

(71) Applicant: University of Massachusetts, Boston, MA (US)

(72) Inventors: Victor Ambros, Hanover, NH (US); Catherine H. Sterling, West Boylston, MA (US)

(21) Appl. No.: 14/493,079

(22) Filed: Sep. 22, 2014

Related U.S. Application Data
(60) Provisional application No. 61/880,566, filed on Sep. 20, 2013.

(51) Int. Cl. C12N 15/10

(52) U.S. Cl. CPC C12N 15/1096
USPC 506/26

(57) ABSTRACT

This disclosure provides methods and compositions for generating cDNA libraries.

### CLAIM OF PRIORITY

This application claims priority under 35 USC §119(e) to U.S. Patent Application Ser. No. 61/880,566, filed on Sep. 20, 2013. The entire contents of the foregoing are hereby incorporated by reference.

### TECHNICAL FIELD

This disclosure generally relates to methods and compositions for generating cDNA libraries.

### BACKGROUND

Understanding the trancriptome is integral to understanding development and disease. Various technologies, including sequenced-based approaches, have been developed to identify and quantify both coding and noncoding RNA. Short (approximately 20-400 bp) reads for sequencing can be generated from long RNAs, such as messenger RNA (mRNA), through fragmentation, or from inherently short RNAs, such as microRNA (miRNA). While sequenced-based approaches have several advantages over hybridization and qRT-PCR-based methods, they are limited in their ability to reliably generate cDNA libraries from small amounts of starting RNAs.

A novel method for generating cDNA libraries from unprecedented low quantities (LQ) of RNA is described herein. Using this method, cDNA libraries can be generated from a range of biofluids as well as from limited tissue volumes isolated from size-limited pathology specimens including biopsy tissue blocks, from small numbers of pure populations of cells isolated from laser capture microdissection of heterogeneous cell populations, and from small numbers of model-organisms.

### SUMMARY

Methods and compositions for generating cDNA libraries are described, including highly sensitive low quantity (LQ) cloning methods for the generation of cDNA libraries from very small quantities of RNA (e.g., pg and sub-pg range) isolated from clinical samples such as samples of human blood plasma. The methods can incorporate one or more novel components including (i) the reduction of gel purification steps, (ii) seamless transition between ligation and RT using sequential reactions in a single tube, and (iii) incorporation of biotinylated nucleotides in the RT reaction to permit efficient purification of cDNA prior to PCR.

In one aspect, a method of preparing a cDNA library from RNA molecules in a biological sample is provided. Such a method typically includes providing a 3′ DNA adapter annealed to a unique DNA oligonucleotide, wherein the unique DNA oligonucleotide comprises a first portion that is complementary to the 3′ DNA adapter and a second portion; ligating the 3′ DNA adapter to RNA molecules, wherein the ligating is performed under conditions that optimize the ligation reaction; reverse transcribing the RNA molecules in the presence of at least one labeled nucleotide to produce labeled cDNA molecules, wherein the reverse transcribing is performed under conditions that optimize the reverse transcription; circularizing the labeled cDNA molecules, wherein the circularizing is performed under conditions that optimize the circularization reaction; optionally purifying and/or isolating the cDNA molecules; linearizing and performing a first amplification of the cDNA molecules; and purifying the amplification product, thereby preparing a cDNA library. In some embodiments, the methods further include performing a second amplification of the product from the first amplification to add platform-specific adapters.

In some embodiments, the unique DNA oligonucleotide further comprises a randomer, e.g., comprising or consisting of 2-20 random nucleotides, e.g., 2-12, 2-8, 4-8, or 4-6 random nucleotides. In some embodiments, the RNA molecules are total plasma RNA molecules. In some embodiments, the RNA molecules are small, circulating RNA molecules. In some embodiments, the RNA molecules are microRNA molecules.

In some embodiments, the conditions that optimize the ligation reaction include carrying out the reaction in the presence of at least a 5:1 molar excess ratio of the 3′ DNA adapter:RNA molecules. In some embodiments, the conditions that optimize the ligation reaction include carrying out the reaction for 6 hours at 30° C. In some embodiments, the labeled nucleotide is a biotinylated labeled nucleotide.

In some embodiments, the reverse transcribing step is performed in the presence of two labeled nucleotides. In some embodiments, the conditions that optimize the reverse transcription reaction include a shorter reaction time relative to manufacturer's instructions. In some embodiments, the conditions that optimize the reverse transcription reaction include carrying out the reaction in the presence of significantly less enzyme relative to manufacturer's instructions. In some embodiments, the conditions that optimize the reverse transcription reaction include carrying out the reaction at a temperature that avoids denaturation of the 3′ DNA adapter and the unique DNA oligonucleotide.

In some embodiments, the methods further include removing the RNA molecules and precipitating the labeled cDNA molecules following the reverse transcribing step.

In some embodiments, the conditions that optimize the circularization reaction include carrying out the reaction in the presence of 20% of the recommended amount of enzyme. In some embodiments, the enzyme is CircLigase I or CircLigase II single-stranded DNA ligase (Epicenter). In some embodiments, the conditions that optimize the circularization reaction include carrying out the reaction in the presence of betaine.

In some embodiments, the actions include purifying and/or isolating the circularized cDNA molecules. In some embodiments, the purifying step is a gel purifying step. In some embodiments, the purifying step further comprises a size selection step. In some embodiments, the isolation is by streptavidin-labeled beads.

In some embodiments, the unique DNA oligonucleotide comprises at least one ideoxyU nucleotide and the linearizing step is performed using UDG.

In some embodiments, the biological sample is selected from the group consisting of biofluids, biopsy tissue blocks, and cells isolated from laser capture microdissection. In some embodiments, the biological sample comprises about 1 pg or less of total RNA.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-C. Overview of 3-day generation of cDNA libraries. (A) On the first day, total RNA is ligated to a 3′ adapter and cDNA is generated by reverse transcription by tandem reactions in a single tube, RNA is degraded and cDNAs are isolated by ethanol precipitation. (B) On the second day, cDNAs are circularized, size selected by gel fractionation and eluted overnight in the presence of streptavidin beads. (C) PCR is done on bead-bound purified cDNAs to generate templates ready for high-throughput sequencing.

FIGS. 2A-G. Detailed LQ cloning method. (A) A pre-adenylated (rApp) 3′-terminal dideoxy-C(ddC) blocked adapter (gray) is annealed to a ssDNA reverse transcription (RT) oligo (black) in a 1:1 molar ratio. The annealed adapter is ligated to 3′-hydroxyl-containing RNA (orange) using T4 RNA Ligase 2 (truncated K227Q) without ATP. Each RT oligo contains a 5′ Guanine (G) followed by a 4 or 6 nucleotide randomer (NX), a 3-6 nucleotide barcode (BAR) and 3 internal deoxyUridine (dU) nucleotides. The adapter::RT oligo hybrid is in excess over RNA, resulting in free adapter::primer material present in the completed reaction. (B) Reverse transcription of ligated RNA is carried out in the same tube as the ligation reaction generating ‘+ insert’ and ‘no insert’ cDNA products (red and black line) using dGTP, dTTP, dATP, dCTP as well as biotinylated dATP and dCTP (yellow ‘B’-containing circles). The RNA template is degraded (dashed orange line) by base hydrolysis and cDNA is ethanol precipitated with ammonium acetate to facilitate maximum removal of free adapter and unincorporated nucleotides (C). Ethanol precipitated cDNAs are circularized (D) and resolved on a 10% denaturing polyacrylamide gel. ‘+ insert’ circularized cDNAs are isolated by excising and eluting them from the gel overnight in the presence of magnetic streptavidin beads (E). Bead-bound ‘+ insert’ cDNAs serve as templates in the first round of PCR. Amplification is done using a mix containing uracil-N-deglycosylase (UNG) to remove dU nucleotides, thereby generating a linear template through strand scission, and with primers complimentary to the 3′ adapter (blue) and 5′ end of the RT oligo (tan) (F). First round PCR products are resolved on an 8% native polyacrylamide gel, the 60-70 nucleotide products are excised and a portion is used as the template for second round PCR. Second round PCR products are generated using primers complimentary to the 3′ adapter (dark blue) and 5′ end of the RT oligo (brown) that contain the full Illumina or Ion Torrent adapter sequences (dark blue and brown) (G).

FIG. 3. Computational pipeline for analysis of deep sequencing libraries. See Materials and Methods section for detailed explanation.

FIGS. 4A-B. LQ and 2-linker cloning methods isolate small RNAs from a synthetic miRNA mixture and total RNA from human blood plasma. (A) The frequency of small RNA read lengths obtained from libraries generated with decreasing amounts of the synthetic miRNA mixture using the LQ method (black text) and the 2-linker method (gray line, red text). Library designation indicated in ( ). Where multiple libraries are indicated, the distribution is shown as the average of these libraries. (B) The frequency of small RNA read lengths in libraries generated using total RNA isolated from human blood plasma is represented as the average across all plasma libraries with error bars indicating the standard deviation.

FIGS. 5A-D. Evaluation of LQ cloning method accuracy. Histograms representing the profile of the nucleotide at the 5′ end (A) and 3′ end (B) of reads mapped to the LT-miRmix in each library using the LQ method (black text) and 2-linker method (red text). LQ library made with heat prior to the RT is shown in brown text and LQ library made without biotin is shown in purple text. (C) The frequency of reads cloned versus combined total G/U content of reads using the LQ method (black text) and the 2-linker method (red text, gray line). Library designation indicated in ( ). Where multiple libraries are indicated, the distribution is shown as the average of these libraries. (D) Histogram representing the percentage of Guanine (G) and Uracil (U) mutated in reads mapped to the LT-miRmix subset reference in libraries generated using either the LQ method (black text) or 2-linker method (red text).

FIGS. 6A-B. Examination of 5′ and 3′ end variation in miRNA mixture cloned sequences from the LT-miRmix. (A) Table demonstrating reverse transcription reaction conditions and corresponding average percentage of 5′ terminal additions computed on LT-miRmix subset reference (see Materials and Methods section). Libraries D1 and D2 (Development 1 and Development 2, respectively) represent libraries examined early in method development with varied RT reaction conditions as indicated. (B) The frequency of small RNA read length for those miRNAs with a fixed 5′ end indicating 3′ end variation across examined libraries. Library designation indicated in ( ). Where multiple libraries are indicated, the distribution is shown as the average of these libraries.

FIGS. 7A-E. Analysis of synthetic miRNAs captured by the LQ cloning method. Deep sequencing data of an equimolar mixture of 29 synthetic miRNAs (29-miRmix) (23) before (gray bars) and after (blue bars) removal of sequences generated by PCR hotspots. The proportion of total reads (Y axis) for each miRNA (X axis) is plotted. The amount of input RNA (2.5 pmol-500 amol) and 3′ adapter (10 pmol or 1 pmol) are indicated. All 3′ adapters were pre-annealed to an equal concentration of RT oligo (see Materials and Methods section) and all RT oligos have a 4 nt randomer sequence. Dashed line represents the expected result for equimolar sequence representation and miRNAs with ≧50% G/C content are indicated with red text. Legend for FIGS. 7A-D is next to 7E.

FIG. 8. Comparison between cDNA libraries generated from the synthetic miRNA mixture. Correlation coefficients (R) were computed using read counts associated with each sequence in the LT-miRmix reference set. Libraries were sequenced on the Ion Torrent (yellow) or Illumina (orange) platform. Dark blue boxes highlight correlations between libraries generated from decreasing amount of RNA and sequenced on either the Ion Torrent or Illumina platform. Light blue boxes highlight correlations between libraries made from identical input material and sequenced on both the Ion Torrent and Illumina sequencing platforms. Brown box highlights correlations between libraries made from identical input material with (10A) or without heat (10B) prior to the RT. Purple box highlights correlations between libraries made from identical input material with (10C) or without (10D) Biotin incorporation. Correlations between libraries made with 9.1 or 0.9 pmol of input using the 2-linker cloning method (red text) are highlighted in the red box.

FIGS. 9A-C. Scatter plots comparing miRNA reads from different library preparations and sequencing methods. (A) Comparison of miRNA reads generated by LQ cloning method originating from 50-fold difference in input RNA concentration. (B) Comparison of miRNA reads generated by 2-linker cloning method originating from 10-fold dilution of input RNA concentration. (C) Comparison of miRNA reads generated by LQ method versus 2-linker method. R=correlation coefficient between the two compared libraries.

FIG. 10. Small RNAs identified in human blood plasma. Total small RNA content in cDNA libraries generated from human blood plasma.

### DETAILED DESCRIPTION

Current methods for creating small RNA-derived cDNA libraries for deep sequencing requires quantities of total RNA in excess of hundreds of picograms (pg) and involves multi-step procedures that are unsuitable for use in the clinical setting, where sample material is limited, and for high throughput of many samples. Moreover, the inevitable and significant loss of material at each procedural step, largely due to gel purification steps, which can result in combined losses of up to 40% of the starting material, renders these approaches impractical for working with the very small amounts of RNA available from certain samples such as patient samples.

Methods are described herein that efficiently and reproducibly clone circulating RNAs, including miRNAs, from body fluids or tissue samples for optimal generation of cDNA libraries from small RNAs. Significantly, data using the methods described herein confirms efficient, successful, and reproducible generation of libraries using less than 10 picograms (pg) of total RNA, which is at least 1,000 fold less input than that required by existing methods while still obtaining a similar profile of expressed miRNAs. Unlike existing methods, the methods described herein are able to create cDNA libraries from very low input volumes and concentrations and, thus, have broad applications in clinical settings.

The present methods can be used for generating cDNA libraries from unprecedented small quantities of RNA is described herein. Using this method, cDNA libraries can be generated from a range of biofluids as well as from limited tissue volumes isolated from size-limited pathology specimens including biopsy tissue blocks, from small numbers of pure populations of cells isolated from laser capture microdissection of heterogeneous cell populations, and from small numbers of model-organisms.

In some embodiments, the methods described herein can be used to efficiently and reproducibly clone circulating miRNA from small volumes of human body fluids. This streamlined protocol optimizes the generation of miRNA cDNA libraries by eliminating nearly all gel purification steps and by dramatically streamlining several other steps including:

i) isolation of small RNA;

ii) linker ligation to cDNA synthesis transition;

iii) isolation of RNA-derived cDNA;

iv) conversion of circularized cDNA into PCR-amplified libraries; and

v) ability to multiplex samples for high-throughput analysis.

Importantly, data using the approach described herein confirms efficient, successful, and reproducible generation of libraries using less than 1 picograms (pg) of total RNA: at least 1,000-fold less than other approaches, while finding a similar profile of expressed miRNAs. Unlike current methods, the methods described herein meet the challenge of creating cDNA libraries from very low input volumes and concentrations and has broad applications in a clinical setting.

Briefly, in some embodiments the present methods includes one or more of the following features:

a) eliminates small-RNA size selection from total RNA by optimizing placement of the size selection at later step (see “j” below).

b) strategically places barcode and randomer in the RT oligo to facilitate multiplexing of samples and ability to identify PCR hotspots while minimizing ligation-introduced biases.

c) uses pre-annealed 3′ adapter::RT oligo to facilitate consistent and reproducible concentration of 3′ linker and the ratio of 3′ linker to RT oligo in the ligation reaction.

d) eliminates ligation product size selection by transferring from ligation reaction directly to RT reaction through incorporation of an optimized RT reaction buffer.

e) provides rapid and efficient RT reaction by using pre-annealed 3′ linker::RT oligo and keeping enzymatic reaction temperatures below that of the annealing temperature for annealed substrates.

f) minimizes RT terminal-transferase activity through optimization of: i) RT enzyme selection, ii) RT enzyme concentration, and iii) RT reaction time.

g) provides for efficient recovery of desired cDNA away from unwanted linken:RT primer cDNA through incorporation of biotinylated nucleotides into cDNA derived only from the RNA::3′ linker ligation product.

h) eliminates cDNA size selection by circularizing the entire pool of cDNAs generated from the RT reaction.

i) provides for efficient circular ligation of cDNA by including nucleic acid carrier in the circular ligation reaction by circularizing the entire pool of cDNAs generated from the RT reaction.

j) strategically places size selection step after circularization of cDNA thereby maximizing separation on the gel and enabling the ability to isolate multiple fractions and/or RNA species cloned.

k) provides for efficient isolation of desired biotinylated cDNA product by eluting circularized product from the 10% PAGE gel in direct presence of streptavidin beads, thereby eliminating (through bead washing) all unwanted, non-biotinylated, material.

l) provides for efficient amplification of material by performing Round 1 PCR directly on the streptavidin beads, thereby eliminating sample loss due to inefficient nucleic acid elution from streptavidin beads.

m) efficiently linearizes circular cDNA via Uracil DNA Glycosylase (UDG)-mediated removal of ideoxyU; the 3×(ideoxyU) in the RT oligonucleotides are removed by UDG contained in the Round 1 PCR master mix.

n) provides for efficient amplification of Round 2 PCR products by using PAGE gel slices from the Round 1 PCR as template in the Round 2 reaction. The improved procedures described herein can significantly overcome deficiencies exhibited by current methods for cloning small RNA. Namely, in order to compensate for significant material losses, current methods for cloning RNA from human plasma samples requires at least 1,000-fold more starting material than the methods described herein. These losses occur in current methods as a result of multiple inefficient sample recovery steps, such as those involving size selection and material elution from PAGE gels, enzymatic ligation and/or restriction digestion reactions, as well as necessary sample concentration and buffer exchanges.

Through the use of pre-annealed cloning substrates, strategic placement of material size selection, incorporation of biotinylated nucleotides specifically into cDNA generated from cloned RNA, utilization of ideoxyU and UDG for linearization of material in the first round of PCR, as well as other steps clearly described herein, the methods described herein to generate cDNA libraries allows for the unprecedented ability to clone from exceedingly small and limited amounts of material.

In some embodiments, the methods described herein start with pre-annealing a 3′ DNA adapter to a unique DNA oligonucleotide Annealing conditions are known in the art and generally depend upon the length of the sequences being annealed as well as the nucleotide composition of the sequences. As used herein, a 3′ DNA adapter refers to an oligonucleotide having a known sequence, and can be virtually any length provided it is long enough to provide binding specificity to a complementary oligonucleotide (for the reverse transcriptase step described below) but not long enough that it inhibits the ligation reaction or the overall method described herein. Without limitation, a first DNA adapter can be between 8 nucleotides (nt) in length and 25 nt in length (e.g., between 8 and 20 nt, between 10 and 20 nt, between 10 and 18 nt, between 12 and 15 nt, or between 14 and 18 nt in length).

As used herein, a unique DNA oligonucleotide has a sequence that includes a first portion that is complementary to and anneals with the 3′ DNA adapter and a second portion. The second portion includes at least a unique “barcode” sequence. Barcode sequences are known in the art and typically refer to a short nucleic acid (e.g., 2, 3, 4, 5, 6, or more base pairs in length) that serves as a unique identifier (e.g., a fingerprint) that can be used to label one or more sequences. The second portion of the unique DNA oligonucleotide also can include additional sequences such as, without limitation, a randomer sequence, which is useful for detecting PCR hotspots, and/or a cleavage sequence. Cleavage sequences are known in the art and include, for example, the use of at least one ideoxyU nucleotide or the use of restriction enzyme sites.

Once the 3′ DNA adapter and the unique DNA oligonucleotide are pre-annealed, the complex (i.e., the 3′ DNA adapter portion of the complex) is ligated to RNAs in a sample. RNA molecules can be obtained from total plasma; for example, RNA molecules can be small, circulating RNA molecules and/or miRNA molecules. It was determined that the efficiency of the ligation reaction (e.g., both capture of the RNA species and the actual ligation reaction) could be improved by carrying out the reaction, for example, in the presence of at least a 5:1 molar excess ratio of the 3′ DNA adapter:RNA molecules. It also was determined that the efficiency of the ligation reaction can be improved by incubating the reaction for about 6 hours at about 30 C.

The RNA molecules then are reverse transcribed in the presence of one or more (e.g., two, three or four) labeled nucleotides to produce labeled, single-stranded cDNA molecules. Labeled nucleotides are used routinely in the art, and include, without limitation, biotin (e.g., biotin-16-AA-2′dCTP, biotin-11-dATP) or any other labeled nucleotide that can be incorporated into cDNA and that contain a ligand that can be used for subsequent affinity purification. It was found that the efficiency of the reverse transcription reaction can be improved by using a shorter reaction time relative to the manufacturer's instructions (e.g., 5 mins instead of 30-60 mins), using significantly less enzyme relative to the manufacturer's instructions (e.g., 0.5 units instead of 200 units), and/or using a reaction temperature that is below the melting temperature (Tm) of the 3′ DNA adapter sequence and the complementary portion of the unique DNA oligonucleotide.

After the cDNA molecules are produced, the RNAs within the DNA:RNA hybrid are removed and the labeled cDNA molecules are precipitated to remove any enzymes, reagents or free nucleotides (i.e., ribonucleotides and deoxyribonucleotides). Methods of removing RNA molecules from a DNA:RNA hybrid are known in the art and include, without limitation, enzymatic degradation (e.g., RNAse H) or exposure to high pH. Methods of precipitating nucleic acids also are well known and used routinely in the art.

Next, the single-stranded labeled cDNA molecules are circularized. Circularization of single-stranded cDNAs is known in the art, and typically utilizes circular DNA ligases such as CircLigase I and II (Epicenter). It was determined herein that 25% of the recommended amount of enzyme was suitable for efficient circularization. In addition, carrying out the reaction in the presence of betaine also improved the circularization reaction while limiting nucleotide bias.

The labeled circularized products then are selectively purified and/or isolated. Methods of purifying nucleic acids (e.g., circularized cDNAs) are known in the art and include, for example, purification from a gel, or purification using membrane separation techniques. Methods of isolating nucleic acids (e.g., circularized cDNAs) are known in the art and generally rely upon, for example, binding the labeled nucleotide(s). For example, if the label is biotin, then an isolation step can utilize streptavidin (e.g., streptavidin-bound beads) to bind the biotinylated nucleotide(s). It would be understood by those skilled in the art that purification and isolation can be performed as separate and distinct steps, or purification and isolation can be performed simultaneously (or essentially simultaneously). Significantly, a purification and/or isolation step also can include a selection step based on size, such that only cDNAs of a particular size are sequenced.

After purification and/or isolation, the cDNAs are linearized and amplified. The linearizing step can be performed using UDG (e.g., in the presence of at least one ideoxyU nucleotide) or one or more restriction enzymes. Amplifications are well known in the art and include, without limitation, the polymerase chain reaction (PCR) and numerous variations thereof. Simply by way of example, see U.S. Pat. Nos. 4,683,195 and 4,683,202.

The amplification products are purified and are ready for sequencing using any of the existing commercial platforms such as, for example, Illumina, Ovation, or Ion Torrent. In some embodiments, however, a second amplification can be performed, for example, to add platform-specific adapters.

As discussed above, the methods described herein are particularly suitable for small sample sizes. Although not limited to small samples, representative biological samples include biofluids, biopsy tissue blocks, and cells isolated from laser capture microdissection. Significantly, the methods described herein can be effectively and reliably performed on biological samples that contain 10 pg or less of total RNA.

In addition to the methods described herein, articles of manufacture (e.g., kits) are provided herein. It would be understood that any number of enzymes and/or reagents can be provided in an article of manufacture in one or more containers, vials, or the like. For example, an article of manufacture can include any or all of the following components: 3′ DNA adapter, unique DNA oligonucleotide, ligase enzyme, ligation buffer, reverse transcriptase, labeled nucleotides, reverse transcription reagents (e.g., buffers, primers, nucleotides), circularization enzyme, circularization buffer, amplification enzymes, amplification reagents (e.g., buffers, primers, nucleotides), and/or beads (e.g., magnetic beads). In addition, instructions for using the article of manufacture can be provided (e.g., in written materials) or directions for obtaining such instructions can be provided (e.g., an address for a website).

In accordance with the present invention, there may be employed conventional molecular biology, microbiology, biochemical, and recombinant DNA techniques within the skill of the art. Such techniques are explained fully in the literature.

### EXAMPLES

The invention will be further described in the following examples, which do not limit the scope of the methods and compositions of matter described in the claims.

### Example 1

### Pre-Annealing the 3′ End Adapter (Modban) and the RT Oligonucleotides

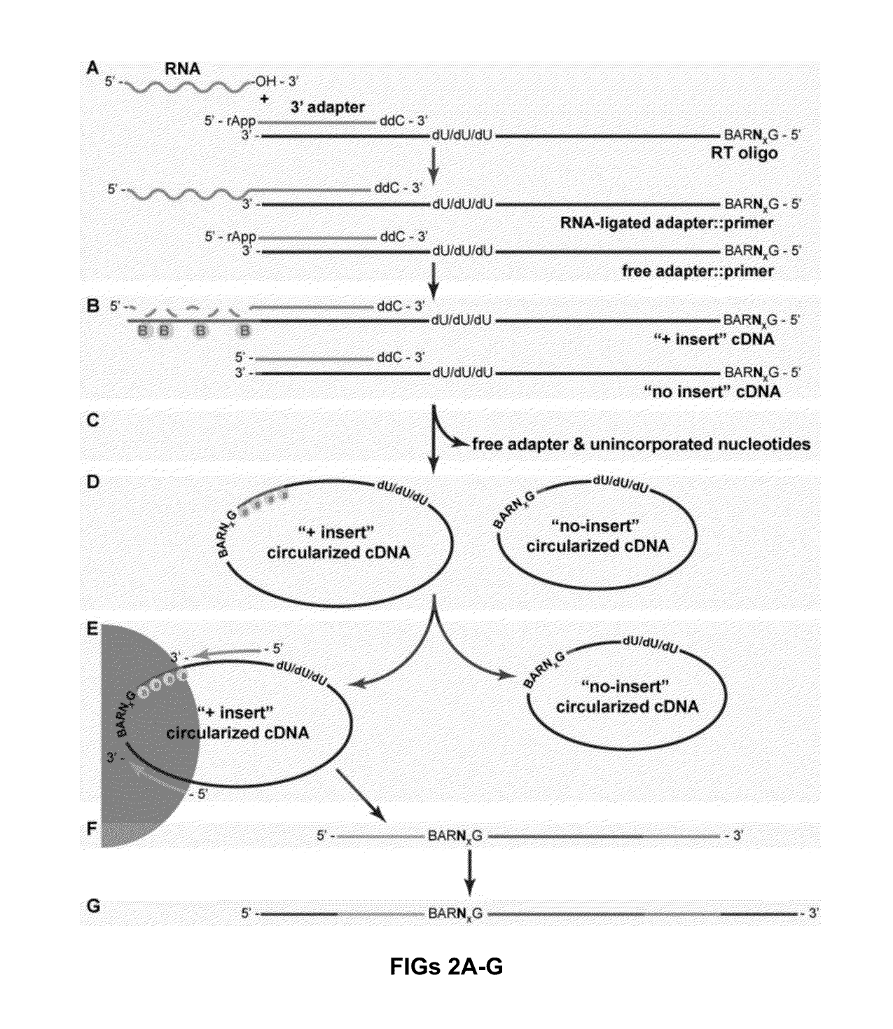

To anneal the 3′ end adapter (App CTG TAG GCA CCA TCA AT ddC (miRNA cloning linker-1 from Integrated DNA Technologies) (SEQ ID NO:1)), referred to as “modban”, with a unique RT oligo (see FIG. 2), 10 μM each of modban and RT oligo were combined in a 20 μl reaction as follows: 5.0 μl 20 μM modban, 5.0 μl 20 μM RT oligo, 2.0 μl 10× annealing buffer, and 8.0 μl RNA milliQH₂O (RNase free ultrapure H₂O, Millipore Biocel). The final concentration of the modban and the RT oligos in the reaction was 5 pmol/μl. The 10× annealing buffer includes 100 mM Tris-HCl pH 7.5, 500 mM NaCl and 10 mM EDTA, and can be made ahead of time and stored at room temperature.

The annealing reaction was performed in a PCR machine. The sample was heated to 95° C. and then cooled at 1° C./min down to 10° C. using a heated lid (105° C.). One pmol of the annealed product was run on an 8% non-denaturing polyacrylamide gel (29:1) to confirm annealing.

### Example 2

### Ligation Reaction with Total Plasma RNA and the Pre-Annealed Product

Total plasma RNA was ligated to the pre-annealed modban:RT oligo product generated in Example 1 as shown in Table A and incubated for 6 hrs at 30° C.

TABLE A

| | | |
|---|---|---|
| 1.0 | μl | 5 pmol/μl pre-annealed modban::RT oligo |
| 50-1000 | amol | total Plasma RNA (<1.0-10 pg total RNA) |
| 1.0 | μl | 10x Ligation reaction buffer |
| 1.5 | μl | DMSO |
| 1.0 | μl | T4 RNA Ligase 2, truncated, K227Q (Such as NEB |
| | | M0373S or M0351S) |
| | | RNA milliQ H2O |
| 10 | μl | total reaction volume |

For optimal ligation, the minimum total plasma RNA should include ≧100 small RNA. The 10× Ligation reaction buffer includes 500 mM Tris-HCl pH 7.5, 100 mM MgCl2, 100 mM DTT and 1 mg/μl BSA. 10× ligation reaction buffer can be made ahead of time and stored in aliquots at −20° C.

### Example 3

### Reverse Transcription Reaction

The ligation product generated in Example 2 was reverse transcribed in a 20 μl reaction that included a 10 μl ligation reaction, 5.0 μl 4×RT reaction buffer and 5.0 μl 4×RT master mix. The 4×RT reaction buffer includes 100 mM Tris-HCl pH 8.5 and 300 mM KCl, and can be made ahead of time and stored at room temperature or 4° C., and the 4×RT master mix includes the remaining components of the RT reaction as shown in Table B, and is made fresh.

TABLE B

| Amount | | Component | Final Concentration |
|---|---|---|---|
| 0.5 μl 10 mM | | 10 mm dGTP | 0.25 mM dGTP |
| dNTP | | 10 mm dTTP | 0.25 mM dTTP |
| | | 6.5 mM dCTP | 0.1625 mM dCTP |
| | | 7.0 mM dATP | 0.175 mM dATP |
| 1.75 | μl | 1.0 mM Biotin-dCTP (TriLink | 0.0875 mM Biotin- |
| | | Biotin-16-AA-2′dCTP, | dCTP |
| | | Cat. # N-5002-1) | |
| 1.5 | μl | 1.0 mM Biotin-dATP (Metkinen | 0.075 mM Biotin- |
| | | Biotin-11-dATP, Cat. # 303-71) | dATP |
| 0.5 | μl | RNase inhibitor | |
| 0.5 | units | Terminal-transferase minus RT | |
| | | enzyme (such as Invitrogen | |
| | | Superscript III Reverse | |
| | | Transcriptase cat. # 18080093) | |
| 0.25 | μl | RNA milliQ H2O | |

The reverse transcription reaction was performed in a PCR machine having a 105° C. heated lid under the following conditions: 45° C. for 5 min and 85° C. for 5 min.

### Example 4

### RNA Removal and Ethanol Precipitation

Any excess RNA was removed by adding 1.8 μl 1M NaOH (at room temperature) to each reverse transcription reaction, incubating for 20 min at 98° C. (in a PCR machine with 105° C. heated lid), and then adding 1.8 μl 1M HCl (at room temperature).

Each reaction was brought up to 200 μl with RNA milliQ H₂O and 2.0 μl polyacryl carrier (Molecular Research Center, Catalog #PC 152), 40.0 μl 10M CH₃COONH₄ (2.0 M final concentration; at room temperature), and 3× volume cold 100% ethanol were added.

The reaction was incubated 1 hour to overnight at room temperature, centrifuged for 30 min at 16,000 rpm at room temperature. The supernatant was removed, the pellet was washed with 200 μl cold 80% ethanol, and the sample was centrifuged for 15 min at 16,000 rpm at room temperature. The supernatant was removed, the pellet was washed with 200 μl cold 70% ethanol, and centrifuged for 15 min at 16,000 rpm at room temperature. The pellet was briefly air-dried and resuspended in 6.25 μl RNA milliQ H₂O.

### Example 5

### Circular Ligation

Circular ligation of the reaction products obtained after the precipitation described in Example 4 was performed using CircLigase II ssDNA Ligase (Epicentre Catalog #CL9025K) in a reaction volume of 10 μl. 6.25 μl of the ethanol precipitated reverse transcribed reaction products was combined with 3.75 μl of the circular ligation master mix and incubated for 2 hours at 60° C. The components of the circular ligation master mix are shown in Table C.

TABLE C

| | | |
|---|---|---|
| 1.0 μl | 10x reaction buffer | 1X |
| 0.5 μl | 50 mM MnCl2 | 2.5 mM |
| 2.0 μl | 5M Betaine | 1M |
| 0.25 μl | CircLigase II ss DNA ligase (Catalog #CL9025K) | 2.5 U/μl |

### Example 6

### Gel Purification and Size Selection of Circular Ligation Product

The entire reaction from the circular ligation described in Example 5 was run on a 10% denaturing gel. The circular-ligated product was cut out of the gel and transferred to a low-retention 1.7 ml Eppendorf tube. The following was added to each gel slice in the Eppendorf tube: 1200 μl TE and 5.0 μl washed magnetic hydrophilic streptavidin beads (NEB product #S1421S; 5 μl beads washed with 3×50 μl buffer WB (0.5 M NaCl, 20 mM Tris-HCl pH 7.5 and 1.0 mM EDTA)). The samples were shaken at 1100 rpm overnight at room temperate.

### Example 7

### Isolating Biotinylated Product

The supernatant and the beads were transferred to a clean, low retention 1.7 mL eppendorf tube, the tube was magnetized, and the supernatant was removed and discarded. The beads were washed with 3× 1 mL buffer WB, magnetizing and removing the supernatant between each wash, and then resuspended in 10 μl RNA milliQ H₂O.

### Example 8

### First Round of PCR Amplification

For the first round of PCR amplification, 5.0 μl of the bead suspension produced in Example 7 was combined with 25.0 μl master mix. For each reaction, the master mix includes 15.0 μl of 2× TaqMan Gene Expression Master Mix (ABI Catalog #4369016) to a final concentration of 1×10.0 μl RNA milliQ H₂O, and 0.3 μl of a 10 μM solution of each primer (ion P1 short, 5′ GAT CTA CAG TCC GAC GAT C 3′ (SEQ ID NO:2); and ion A short, 5′ ATT GAT GGT GCC TAC AG 3′ (SEQ ID NO:3)), to a final concentration of 0.1 μM. The UDG enzyme for cleavage of the product is provided by the manufacturer in the Master Mix.

The amplification conditions include 2 mins at 55° C., 10 mins at 95° C. and 22 cycles of 95° C. for 15 mins and 55° C. for 60 min, followed by a hold at 10° C. 22 cycles was sufficient in the experiments described herein, but the number of cycles in the first round of PCR amplification may need to be adjusted to optimize the results. A sample of 15 μl was removed at cycle 20 and 22.

All samples were run on 8% non-denaturing polyacrylamide gel (29:1), and the gels were stained using SYBR gold (Invitrogen Catalog #S11494). Bands were visualized on a blue-light transilluminator and the 65 nt PCR product was excised, cut into 2 roughly equal pieces, and stored at −20° C.

### Example 9

### Second Round of PCR Amplification

For the second round of PCT amplification, one-half of the gel slice obtained following the Round 1 PCR Amplification is combined with 100.0 μl master mix. For each reaction, the master mix includes 50.0 μl of 2× AmpliTaq Gold Fast PCR master mix (ABI Catalog #4390941) to a final concentration of 1×50.0 μl RNA milliQ H₂O, and 1.0 μl of a 10 μM solution of each primer (ion P1 long, 5′ CCA CTA CGC CTC CGC TTT CCT CTC TAT GGG CAG TCG GTG ATC TAC AGT CCG ACG ATC 3′ (SEQ ID NO:4); and ion A long, 5′ CCA TCT CAT CCC TGC GTG TCT CCG ACT CAG ATT GAT GGT GCC TAC AG 3′ (SEQ ID NO:5)), to a final concentration of 0.125 μM.

The amplification conditions include 10 mins at 95° C., 8-12 cycles at 95° C. for 15 min and 60° C. at 60 mins, followed by a hold at 10° C.

### Example 10

### Purification of PCR Product

The PCR product was purified away from the primers using EZ-10 Spin Column PCR Purification Kit (BioBasic Inc. Cat. # BS664) and eluted in 30 μl kit-provided EB buffer. The amount of PCR product was quantified using an 8% non-denaturing polyacrylamide gel (29:1) along with a known DNA standard such as Low Molecular Weight DNA ladder (NEB, Catalog #N32335).

### Example 11

### Quality Control Checks

The PCR product was quantified using a NanoDrop machine and yields of 5-20 ng/μl were obtained. However, since NanoDrop readings below 10 ng/μl are not accurate, quantification using another method (e.g., an Agilent 2100 Bioanalyzer High Sensitivity DNA chip, Ion Library Quantification Kit (Life Technologies Catalog #4468802)) is still necessary.

In addition, PCR products can be cloned using, for example, TOPO TA cloning protocol (Invitrogen, Catalog #K450001) and sequenced using, for example, Sanger sequencing or Ion Torrent sequencing.

### Example 12

### Preparing cDNA Libraries from Scarce Biological Samples

In this example, an exemplary highly sensitive LQ cloning method was used for the generation of cDNA libraries from very small quantities of RNA (pg and sub-pg range) isolated from clinical samples of human blood plasma. The method incorporated several novel components including (i) the reduction of gel purification steps, (ii) seamless transition between ligation and RT using sequential reactions in a single tube and (iii) incorporation of biotinylated nucleotides in the RT reaction to permit efficient purification of cDNA prior to PCR.

Materials and Methods

The following materials and methods were used in this Example.

Blood Draw and Plasma Isolation

3×10 ml blood was collected in ethylenediaminetetraacetic acid (EDTA) blood collection tubes and spun at 1100×g for 20 min at 4° C. Plasma from all three tubes was combined into one large cryovial, aliquoted into 1 ml cryovials, and stored at −80° C.

Nucleic Acid Manipulations

1.7 ml siliconized (low retention) microcentrifuge tubes were used whenever possible to facilitate maximum recovery of material. For all reactions done in a PCR machine, either 0.2 ml strip tubes or 0.2 ml 96-well plates were used depending on sample size and number.

Synthetic miRNA Mixtures

Obtained from Life Technologies, referred to as ‘LT-miRmix’, and Rui Yi lab, referred to as ‘29-miRmix’ (23).

Total RNA Isolation

For total RNA extraction from samples of human blood plasma, 250 μl plasma aliquots (stored at −80° C.) were thawed on ice and cleared by centrifugation at 16,000 rpm for 15 min at 4° C. 200 μl of the supernatant was removed and total RNA was extracted using Trizol, followed by extraction with phenol/chloroform, and ethanol precipitation at −80° C. overnight using polyacryl carrier (Molecular Research Center) and 3M KAc. Precipitate was recovered by centrifugation, washed with 200 μl 70% ethanol, resuspended in 10.0 μl RNase free water and stored at −80° C.

Determination of Plasma Equivalents

‘Plasma equivalents’ refer to the relative amount of plasma used to generate cDNA libraries from RNA isolated from human blood plasma. Using the protocol described above, 10 μl of RNA corresponds to approximately 200 μl of plasma.

Quantification of miRNA in Total RNA from Human Blood Plasma

Approximate quantity of miRNA in samples of total RNA isolated from human blood plasma was determined using miR TaqMan real-time qPCR assays (Life Technologies), with reference to a standard curve generated using a known quantity of LT-miRmix. RT was performed using the miR-223 specific stem-loop primer (ID 002295) and the MicroRNA RT Kit (both Life Technologies). Reaction conditions followed manufacturer's instructions: 16° C. 30 min, 42° C. 30 min, 85° C. 5 min. One microliter of the RT reaction was used as template in reactions containing 1× miRNA-specific TaqMan primers/probes in combination with 1× TaqMan GeneExpression Master Mix (Life Technologies) according to the manufacturer's instructions in a total reaction volume of 10.0 μl. Samples were split and run as 3×3.0 μl reactions along with a no template sample as a negative control. PCR reaction conditions followed manufacturer's instructions: 50° C. 2 min, 95° C. 10 min, 40× (95° C. 15 s, 60° C. 1 min). Assays were run on a 7900HT Fast Real-Time instrument (Life Technologies).

Oligonucleotide Substrates, Adapters and Primers

3′ adapter, 5′ adapters, RT oligonucleotides and PCR primers (Tables 1 and 2) were obtained from Integrated DNA Technologies (IDT). RT oligonucleotides were HPLC or PAGE purified by IDT.

Pre-Annealing of Reverse Transcription Primer and 3′ Linker Oligonucleotide

5.0 μl of 20 μM modban 3′ adapter (IDT miRNA cloning linker 1) (Ref. 1) and 5.0 μl of 20 μM RT oligonucleotide (Table 1) were incubated in 20.0 μl total volume with annealing buffer (10 mM Tris-HCl pH 7.5, 50 mM NaCl, 1.0 mM EDTA) Annealing reactions were performed in a thermocycler with a 105° C. heated lid as follows: 95° C. 1 min, cool 1° C. every minute for 85 min.

1.0 pmol of annealed product (3′ adapter::RT oligo) was analyzed on an 8% non-denaturing polyacrylamide gel (29:1) to confirm annealing.

TABLE 1

| | | | | SEQ |
|---|---|---|---|---|
| | 5′ | | 3′ | ID |
| name | mod | Oligo sequence | mod | NO: |
| 3′ adapter | rApp | CTGTAGGCACCATCAAT | ddC | |
| 5′ adapterA | | 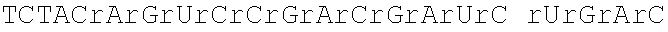 | | |
| 5′ adapter | | 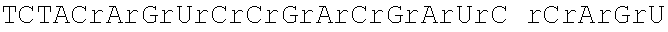 | | |
| bar01 | Phos | 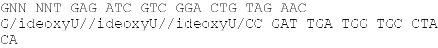 | | |
| bar02 | Phos | 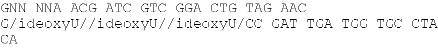 | | |
| bar03 | Phos | 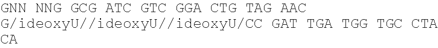 | | |
| bar04 | Phos | 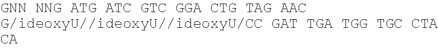 | | |
| bar05 | Phos | 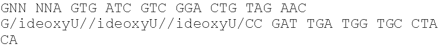 | | |
| bar06 | Phos | 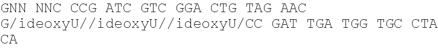 | | |
| bar07 | Phos | 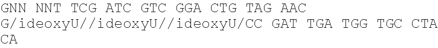 | | |
| bar08 | Phos | 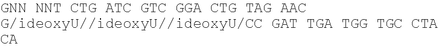 | | |
| bar09 | Phos | 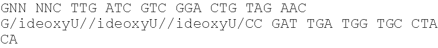 | | |
| bar10 | Phos | 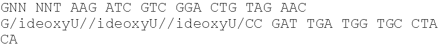 | | |
| bar11 | Phos | 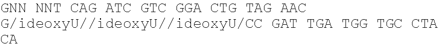 | | |
| bar12 | Phos | 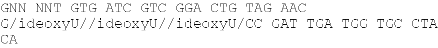 | | |
| bar13 | Phos | 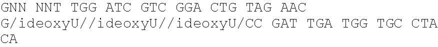 | | |
| bar14 | Phos | 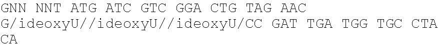 | | |
| bar15 | Phos | 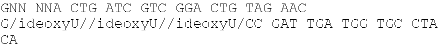 | | |
| bar16 | Phos | 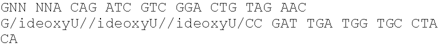 | | |
| bar17 | Phos | 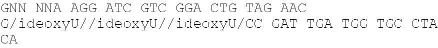 | | |
| bar18 | Phos | 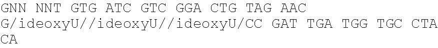 | | |
| bar19 | Phos | 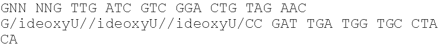 | | |
| bar20 | Phos | 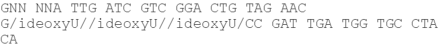 | | |
| bar21 | Phos | 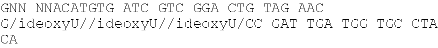 | | |
| bar22 | Phos | 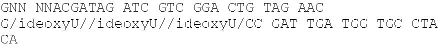 | | |
| bar23 | Phos | 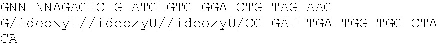 | | |
| bar24 | Phos | 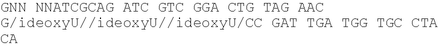 | | |
| bar25 | Phos | 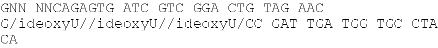 | | |
| bar26 | Phos | 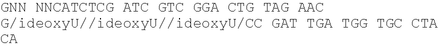 | | |
| bar27 | Phos | 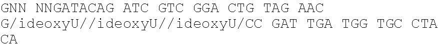 | | |
| bar28 | Phos | 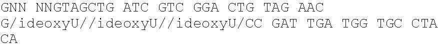 | | |
| bar29 | Phos | 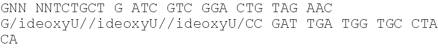 | | |
| bar30 | Phos | 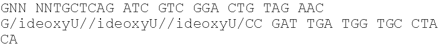 | | |
| bar31 | Phos | 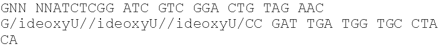 | | |
| bar32 | Phos | 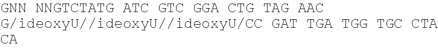 | | |
| bar33 | Phos | 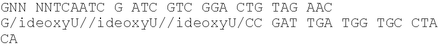 | | |
| bar34 | Phos | 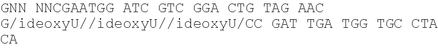 | | |
| bar35 | Phos | 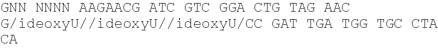 | | |

3′ adapter and reverse transcription (RT) oligos used in ligation and RT reactions. 5′ and 3′ end modifications were as indicated. 5′ adapters contained RNA nucleotides (r) and a 4 nucleotide (nt) barcode (highlighted). RT oligos contained a 4 or 6 nt randomer (NNNN or NNNNNN), a 3-6 nt barcode (highlighted) and 3 internal deoxyUridine (ideoxyU)

3′ Ligation Reactions

5.0 pmol of annealed 3′ adapter::RT oligo product was incubated with various quantities of either a synthetic miRNA mixture or total RNA from human plasma (Table 3). 10.0 μl reactions contained 50 mM Tris-HCl pH 7.5, 10 mM MgCl₂, 10 mM DTT, 0.1 μg/μl BSA, 15% DMSO and 1.0 μl T4 Rn12 truncated K227Q (24). The T4 Rnl2tr K227Q was prepared using Addgene plasmid 14072 as described in (25). Samples were incubated for 6 h at 30° C. in a PCR machine with a 105° C. heated lid.

First-Strand cDNA Synthesis

RT of ligation products was performed directly in the ligation reaction mixture after completion of ligation by addition of the following: 5.0 μl of 4×RT reaction buffer (100 mM Tris-HCl pH 8.0, 300 mM KCl), 5.0 μl of 4×RT master mix (0.25 mM dGTP, 0.25 mM dTTP, 0.1625 mM dCTP, 0.175 mM dATP, 0.0875 mM Biotin-dCTP (Trilink Biotin-16-AA-2′dCTP), 0.075 mM Biotin-dATP (Metkinen Biotin-11-dATP), 20 units RNase inhibitor, 0.5 units Invitrogen Superscript III Reverse Transcriptase, a thermally stable RT enzyme, lacking terminal transferase activity (Life Technologies product literature). Samples were incubated for 5 min at 45° C. followed by 5 min at 85° C. in a thermocycler with a 105° C. heated lid.

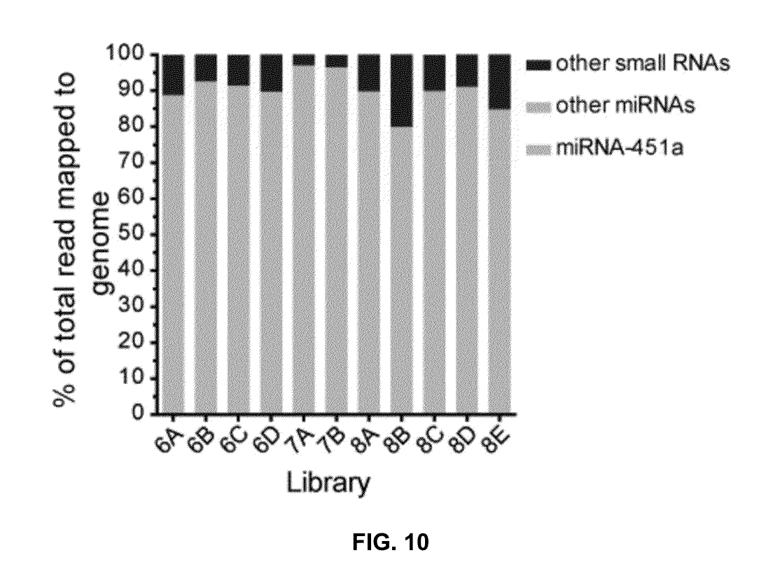

For the LQ-biotin Library (FIG. 10D), cDNA was generated as described above except with 10 mM dNTP containing equal molar concentrations of dGTP, dTTP, dCTP, dATP, and no biotinylated nucleotides.

Removal of Template RNA and Ethanol Precipitation of cDNA Reaction Products

To hydrolyze RNA in the sample after RT, 1.8 μl 1M NaOH was added to each RT reaction, samples were incubated for 20 min at 98° C. in a PCR machine with a 105° C. heated lid and were neutralized by adding 1.8 μl 1M HCl (26).

cDNAs were ethanol precipitated by bringing each reaction up to 200 μl with RNase free H₂O, transferring to a clean, 1.7 ml siliconized (low-retention) microcentrifuge tube, adding 2.0 μl polyacryl carrier (Molecular Research Center), 40.0 μl 10M CH₃COONH₄, 3 volumes 100% ethanol, and incubating at room temperature for a period of 1 h—overnight. Following room temperature incubation, precipitate was recovered by centrifugation at 16,000 rpm for 30 min at room temperature in a microcentrifuge. The supernatant was removed and the pellet was washed with 200 μl 80% ethanol and spun for 15 min at 16,000 rpm at room temperature. The supernatant was removed, the pellet was washed with 200 μl 70% ethanol and spun for 15 min at 16,000 rpm at room temperature. The supernatant was removed and the pellet was resuspended in 10.0 μl circular ligation reaction mix (see below).

Circular Ligation Reactions

Ethanol precipitated cDNA reaction product (see above) was resuspended in 10.0 μl circular ligation reaction mix containing 1.0 μl 1 Ox reaction buffer (0.33M Tris-acetate pH 7.8, 0.66M potassium acetate, 5 mM DTT), 2.5 mM MnCl₂, 1M betaine and 1.25 units CircLigase II ssDNA Ligase (Epicentre). Reactions were incubated at 60° C. for 2 h.

Gel Fractionation, Elution and Isolation of Biotinylated cDNA

Circularized cDNAs and circular single-stranded DNA molecular weight markers were fractionated on separate lanes of a 10% polyacrylamide/7M urea gel. The gel was stained with SYBR Gold (Life Technologies) according to manufacturer's instructions, visualized on a blue light transilluminator and circularized cDNAs migrating in the range of 80-100 nt were excised. Each gel slice was added to a 1.7 ml siliconized (low-retention) microcentrifuge tube containing 400 μl TE+0.3M NaCl. 5.0 μl magnetic hydrophilic streptavidin beads (New England Biolabs) were washed three times with 50 μl buffer WB (0.5M NaCl, 20 mM Tris-HCl pH 7.5, 1.0 mM EDTA), resuspended in 5.0 μl TE+0.3M NaCl and added to each sample. Tubes were shaken overnight at 1,100 rpm at room temperature. After overnight elution, the bead-containing supernatant was transferred to a clean, low-retention 1.7 ml eppendorf tube, tubes were magnetized on a magnetic rack (Life Technologies), supernatants carefully removed, beads washed three times with 1.0 ml buffer WB (magnetizing and carefully removing supernatant between each wash step) and resuspended in 10.0 μl RNase free H₂O.

For the LQ-biotin library (10D), gel fractionation and elution was done as above except magnetic streptavidin beads were excluded. After overnight shaking, buffer containing eluted material was removed, added to a 0.45 μm spin column and spun 3 min at 3,000 rpm at room temperature into a clean, 1.7 ml siliconized (low-retention) microcentrifuge tube. Circularized cDNAs were ethanol precipitated by adding 2.0 μl polyacryl carrier (Molecular Research Center), 1/10 volume 3M KAc pH 5.0, 3 volumes 100% ethanol, and incubating at −80° C. for 1 h. Following −80° C. incubation, precipitate was recovered by centrifugation at 16,000 rpm for 20 min at 4° C. in a microcentrifuge. The supernatant was removed, the pellet was washed with 200 μl 80% ethanol and spun for 15 min at 16,000 rpm at 4° C. The supernatant was removed, the pellet was washed with 200 μl 70% ethanol and spun for 15 min at 16,000 rpm at 4° C. The supernatant was removed and the pellet was resuspended in 10.0 μl RNase free H₂O.

First Round PCR Library Amplification of cDNA Library

PCR amplification was performed using 2× TaqMan Gene Expression Master Mix (Life Technologies) using 50% of the bead suspension from above in a 50.0 μl reaction with a final concentration of 0.1 μM forward and 0.1 μM reverse Round 1 PCR primers (Table 2) for up to 25 cycles removing 10.0 μl every 2 cycles starting at 12 cycles. PCR reactions conditions were: 55° C. 2 min, 95° C. 10 min, 12-25× (95° C. s, 55° C. 1 min), 10° C. hold. Samples were run on an 8% non-denaturing polyacrylamide gel (29:1). The gel was stained with SYBR Gold (Life Technologies) per manufacturer's instructions and visualized on a blue light transilluminator. Appropriate 65-70 nucleotide PCR products were excised at the cycle number when first visible, gel slices cut in half vertically, and stored at −20° C.

TABLE 2
PCR oligonucleotide sequences

| | PCR | | SEQ |
|---|---|---|---|
| Name | round | Sequence 5--3' | ID NO: |
| Ion A | 1 | ATTGATGGTGCCTACAG | |
| short | | | |
| Ion | 1 | GATCTACAGTCCGACGATC | |
| P1 | | | |
| short | | | |
| Ion | 2 | CCATCTCATCCCTGCGTGTCTCCGACTCAG | |
| P1 | | ATTGATGGTGCCTACAG | |
| long | | | |
| Ion A | 2 | CCACTACGCCTCCGCTTTCCTCTCTATGGG | |
| long | | CAGTCGGTGATCTACAGTCCGACGATC | |
| DP3 | 1 | ATTGATGGTGCCTACAG | |
| DP5 | 1 | GTTCTACAGTCCGACGATC | |
| Solex | 2 | ACAGCAGAAGACGGCATACGAATTGATGGT | |
| a 3 | | GCCTACAG | |
| Solex | 2 | AATGATACGGCGACCACCGACAGGTTCAGA | |
| a 5 | | GTTCTACAGTCCGACGAT | |

DNA oligonucleotides used in first (1) and second (2) rounds of PCR amplification for sequencing on the Ion Torrent (indicated by prefix ‘Ion’) or Illumina (DP3, DP5, Solexa 3 and Solexa 5) platforms.

Second Round PCR Amplification of cDNA Library

PCR amplification was performed using 2× AmpliTaq Gold Fast PCR master mix (Life Technologies) using one-half of the first round PCR product gel slice as template in a 100 μl reaction with a final concentration of 0.125 μM forward and 0.125 μM reverse Round 2 PCR primers (Table 2) and 8-12 cycles. Care should be taken to isolate PCR products at low cycle number, before primers are depleted and bulged PCR products appear. (Such bulged products result from denatured products that anneal at the adaptors but not at the center, and appear as a smear migrating slower than the desired PCR product.) An initial 20-30 μl PCR reaction, removing samples at 8, 10 and 12 cycles and resolving on an 8% non-denaturing polyacrylamide gel (29:1), can be performed to determine optimal cycle number. PCR reactions conditions were: 95° C. 10 min, 8-12× (95° C. 15 s, 60° C. 1 min), 10° C. hold.

Purification and Quantification of Second Round PCR Product

Appropriate 116-138 nucleotide second round PCR products were purified using BioBasic Inc. EZ-10 Spin Column PCR Purification Kit following manufacturer's recommendations. Samples were eluted in 30 μl kit-provided elution buffer and quantified using an 8% non-denaturing polyacrylamide gel (29:1) and a known DNA standard (i.e. New England Biolabs Low Molecular Weight DNA ladder) as well as on a high-sensitivity DNA BioAnalyzer chip (Agilent Technologies).

2-Linker cDNA Library Preparation

Libraries were generated as described in Gu et al. (13) with minor modifications. The 3′ ligation was done as described above and 5′ adapters contained the 3′ barcode as described in Table 1.

Sample Preparation and High-Throughput Sequencing

PCR products were sequenced on the Ion Torrent PGM, Ion Torrent Proton or Illumina HiSeq2000 instrument according to manufacturer's protocols.

Computational Pipeline

FastQ file formats contained the following data formats:

Ion Torrent sequences: (3′) adapter-sequence read-GN_{X}-barcode-adapter (5′)

Illumina sequences: (5′) barcode-N_{X}C-sequence read-adapter (3′)

For each library the following steps were applied:
1. Adapters were removed using the Cutadapt method (version 1.2.1) (27): using the -e 0.25 option and the following adapter sequences:
   For Ion Torrent sequences: -g ATTGATGGTGCCTACAG and -a GATCGTTCGGACTGTAGATC
   For or Illumina sequences: -a CTGTAGGCACCATCAAT
2. Sequences were split into libraries according to barcode.
3. Randomer sequences (N_{X}) were removed and saved in the header line of the fasta file for later PCR hotspot analysis.
4. Reads <17 nt were filtered and removed.
5. For Ion Torrent sequences, reads were reverse complemented.
6. Reads with identical sequences were combined and the combined count was saved.
7. The count of each read was corrected by the ‘PCR hotspot’ correction procedure described below.
8. Plasma and mixture libraries were further processed as follows:
   A. For plasma libraries:
Reads were aligned to a reference sequence (see below) using bowtie (28)
-v 3 -f -B 1 -a --best --strata
Alignments were then filtered based on the length of the read and the number of mismatches as follows: for sequence lengths 17, 18-19, 20-24 or >24: 0, 1, 2 or 3 mismatches were allowed, respectively.
   B. For miRNA mixture libraries:
   1. Reads were aligned to reference sequences (see below) and (2) the reference sequences were aligned to the reads using bowtie (28) -v 3 -f -B 1 -a --best --strata

A reference set for the Life Technologies synthetic mixture (LT-miRmix) libraries was constructed as follows:
1. All sequences that were present in mixture libraries 1-5B (Table 3) were enumerated.
2. All sequences with more than 10 reads in ≧7 libraries were identified. In addition, sequences with more than 50 reads in ≧1 library that were also annotated in the miRNA mixture (Life Technologies, personal communication) were collected. Sequences were combined, resulting in a total of 2,299 sequences.
3. Highly overlapping sequences with some 5′ and 3′ overhangs were clustered based on a 17 nt seed sequence, allowing up to two mismatches.
4. Each cluster was represented by a sequence spanning all sequences within the cluster, resulting in 1,047 sequences.
5. These final 1,047 sequences served as the miRNA mixture reference set.

Defining a sub-reference set of the Life Technologies synthetic mixture (denoted as LT-miRmix subset):

To perform an unbiased analysis of 5′ terminal additions in the LQ method, which could be affected by the natural heterogeneity present in the synthetic miRNA mixture, we identified a set of 154 sequences with a fixed starting point (exhibiting no heterogeneity in the 5′ end) for all the reads. These sequences served as the reference set for analysis of 5′ nt additions, 3′ end variability and biotin-introduced mismatch rate.

The reference set for the equimolar 29-miRNA synthetic mixture was as described in Zhang et al. (23).

Reference sets for the human blood plasma libraries were:
1. The human genome sequence, hg19 downloaded from UCSC (29).
2. A list of annotated sequences from the following resources:
   UCSC browser for rRNA, tRNA, snRNA and scRNA sequences (29).
   piRNA bank (30) for piRNA sequences.
   fRNAdb (31) for yRNA and snoRNA sequences.
   miRBase (32) (Release 20) for pre-miRNA and miRNA mature sequences.
   Analysis of Mapping Results

An in-house developed code was used to analyze the results as follows:
1. For the synthetic miRNA mixture libraries the mapping results of (i) reads to reference sequences and (ii) reference sequences to the reads were combined. (The latter is needed in order to take into account potential 5′ or 3′ terminal additions.) A read count for each reference sequence was assigned by taking into account all reads associated with it and keeping track of the relative mapping alignment of each read for further analysis.
2. For plasma libraries the results of (i) mapping to the human genome sequence and (ii) mapping to small RNA sequences were combined. Reads that mapped to the genome with less mismatches than to a small RNA were assigned as non-small RNA human genome matches. To assign read counts to the miRNA sequences we considered all reads that mapped to a pre-miRNA sequence within −5 to +5 nucleotides of the annotated mature miRNA start according to miRBase. For all the other small RNA species, we included all reads that map to a small RNA reference sequence regardless of the mapping position.

For all comparisons done between libraries, a normalized read count (i.e. reads per million of aligned reads) was used.

Analyzing miRmix Subset Sequences

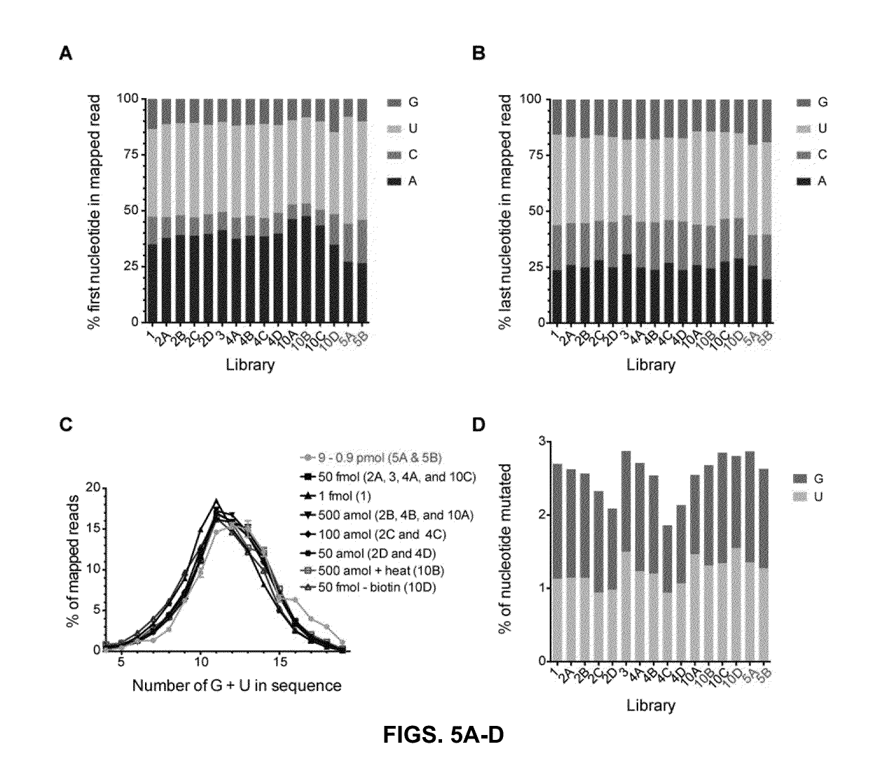

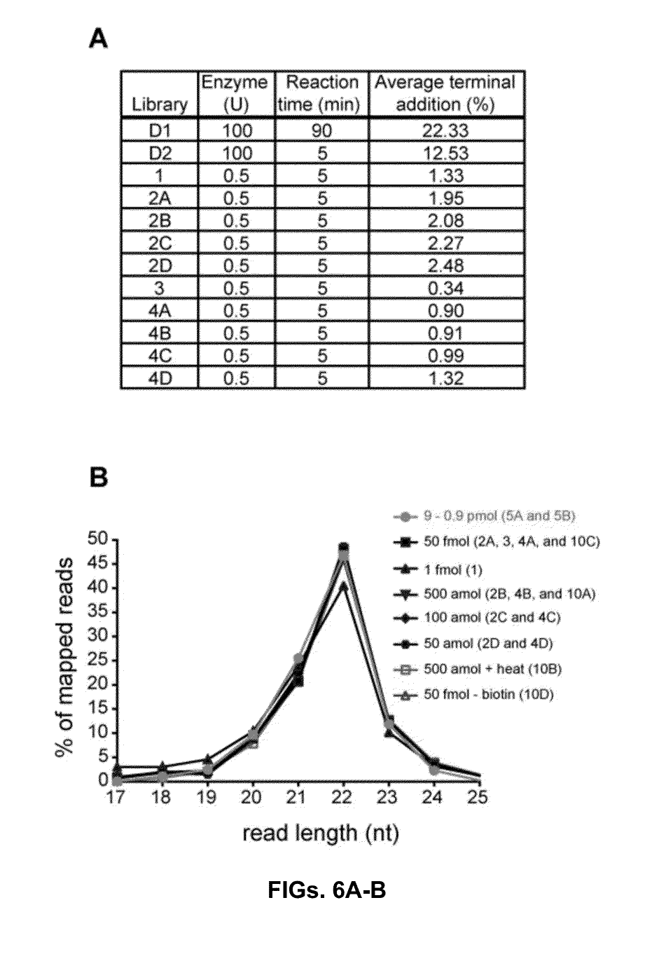

To generate the averages presented in the G/U mutation profile (FIG. 5D), 5′ terminal additions profile (FIG. 6A) and read length profile (FIG. 6B), the synthetic miRNA sequence reference sets were used as follows. For each sequence the following were calculated:

1. The percent of mismatches among the mapped reads (for G or U nucleotides separately).

2. The percent of mapped reads that had a 5′ overhang (of up to 5 nucleotides) beyond the fixed starting point of the sequence.

3. The distribution of read length among the mapped reads.

The averages across all the sequences for each of the measures in points 1-3 are displayed in the figures mentioned above.

PCR Hotspot Correction

For each sequence in a given library, we stored the randomer sequence (N_{X}) associated with it (Step 3 of the computational pipeline). These randomers were used for:
A. Assessing the distribution of randomers from the sequencing data by:
   1. Calculating the distribution of randomers associated with each sequence.
   2. Identifying a set of sequences with random distribution of randomer sequences by:
      a. choosing a set of sequences that has more than 4)(reads and
      b. identifying sequences within (a) where no randomer is represented by >5% of the reads.
   3. Determining the expected distribution of randomers by calculating the average percentage and the standard deviation for each randomer, based on the sequences found in A2. We denote the expected percentage for a randomer i as pᵢ (iε{1 . . . 4×}, Σ4×i=1 p i=100).
B. Identification of PCR hotspots for each combined identical sequence in the library was done by:
   1. Identifying randomers with an observed percentage higher than the expected average plus three standard deviations.
   2. Let i be the randomer identified in B1, with an expected percentage pᵢ (calculated in A3) and an observed count cᵢ. Let n be the total read count observed for the current sequence. The new total count n′ can be corrected to n′=(n−c i)*100(100−p i). The observed count for randomer i is corrected to p i *n′.
   3. Correcting the observed distribution of randomers for all the other randomers based on their c and n′.
   4. Repeating steps B2 and B3 until no corrections are needed or until the total sequence count equals the number of randomers identified for that sequence.

Note: If, for a given sequence, only a subset of randomers was observed, then the expected distribution of these randomers was scaled so that their total summation will be 100, and 0 is assigned in the pᵢ of the other randomers. This step was used to avoid collapsing of reads with underrepresentation of randomers (due to initial low abundance of the read(s)).

TABLE 3

| | | | | | | | | | R1 | R2 | | | | |
|---|---|---|---|---|---|---|---|---|---|---|---|---|---|---|
| | | | | | Quantity | | | | PCR | PCR | | % | % | % |
| | Library | Synthetic | Concentration | | RNA | | Sequencing | Cloning | cycle | cycle | Total read | Filtered | Reads | Reference |
| | ID | mixture | of RNA input | | input (pg) | | platform | method | no. | no. | number | reads | mapped | covered |
| A: | 1 | LT- | 1 | fmol | 6.89 | | Ion Torrent | LQ | 20 | 8 | 363,895 | 3.8 | 82.1 | 99.7 |
| synthetic | | miRmix | | | | | PGM | | | | | | | |
| miRNA | 2A | LT- | 50 | fmol | 344.48 | | Ion Torrent | LQ | 14 | 12 | 438,625 | 0.6 | 85.9 | 98.3 |
| mixture | | miRmix | | | | | PGM | | | | | | | |
| | 2B | LT- | 500 | amol | 3.44 | | Ion Torrent | LQ | 16 | 12 | 350,126 | 2.4 | 81.1 | 99.8 |
| | | miRmix | | | | | PGM | | | | | | | |
| | 2C | LT- | 100 | amol | 0.69 | | Ion Torrent | LQ | 18 | 12 | 348,732 | 7.3 | 58.8 | 99.1 |
| | | miRmix | | | | | PGM | | | | | | | |
| | 2D | LT- | 50 | amol | 0.34 | | Ion Torrent | LQ | 18 | 12 | 274,635 | 7.5 | 61.6 | 98.9 |
| | | miRmix | | | | | PGM | | | | | | | |
| | 3 | LT- | 50 | fmol | 344.48 | | Illumina | LQ | 18 | 10 | 2,479,439 | 6.7 | 95.7 | 97.9 |
| | | miRmix | | | | | HiSeq2000 | | | | | | | |
| | 4A | LT- | 50 | fmol | 344.48 | | Illumina | LQ | 14 | 12 | 2,290,950 | 0.7 | 95.0 | 98.4 |
| | | miRmix | | | | | HiSeq2000 | | | | | | | |
| | 4B | LT- | 500 | amol | 3.44 | | Illumina | LQ | 16 | 12 | 1,946,371 | 3.9 | 86.9 | 99.9 |
| | | miRmix | | | | | HiSeq2000 | | | | | | | |
| | 4C | LT- | 100 | amol | 0.69 | | Illumina | LQ | 18 | 12 | 1,505,284 | 9.9 | 53.1 | 99.5 |
| | | miRmix | | | | | HiSeq2000 | | | | | | | |
| | 4D | LT- | 50 | amol | 0.34 | | Illumina | LQ | 18 | 12 | 608,838 | 7.7 | 51.6 | 99.2 |
| | | miRmix | | | | | HiSeq2000 | | | | | | | |
| | 5A | LT- | 9,100 | fmol | 62,699.00 | | Illumina | 2- | 14 | 8 | 1,718,684 | | 95.2 | 90.9 |
| | | miRmix | | | | | HiSeq2000 | Linker | | | | | | |
| | 5B | LT- | 900 | fmol | 6,201.00 | | Illumina | 2- | 14 | 8 | 2,411,058 | | 94.3 | 92.3 |
| | | miRmix | | | | | HiSeq2000 | Linker | | | | | | |
| | 9A | 29- | 2,500 | fmol | 19,725.00 | | Ion Torrent | LQ | 12 | 10 | 10,644,245 | 6.9 | 92.6 | 100.0 |
| | | miRmix | | | | | proton | | | | | | | |
| | 9B | 29- | 1,250 | fmol | 9,862.50 | | Ion Torrent | LQ | 12 | 10 | 9,693,704 | 5.8 | 93.7 | 100.0 |
| | | miRmix | | | | | proton | | | | | | | |
| | 9D | 29- | 50 | fmol | 344.50 | | Ion Torrent | LQ | 14 | 10 | 6,241,100 | 6.9 | 92.5 | 100.0 |
| | | miRmix | | | | | proton | | | | | | | |
| | 9E | 29- | 1 | fmol | 7.89 | | Ion Torrent | LQ | 22 | 10 | 6,548,076 | 8.1 | 84.4 | 100.0 |
| | | miRmix | | | | | proton | | | | | | | |
| | 9F | 29- | 500 | amol | 3.44 | | Ion Torrent | LQ | 22 | 10 | 1,678,663 | 14.7 | 72.9 | 100.0 |
| | | miRmix | | | | | proton | | | | | | | |
| | 10A | LT- | 500 | amol | 3.44 | | Ion Torrent | LQ | 24 | 10 | 5,347,655 | 7.9 | 89.2 | 95.4 |
| | | miRmix | | | | | proton | | | | | | | |
| | 10B | LT- | 500 | amol | 3.44 | | Ion Torrent | LQ + | 24 | 10 | 2,346,229 | 9.3 | 87.3 | 93.4 |
| | | miRmix | | | | | proton | heat | | | | | | |
| | 10C | LT- | 50 | fmol | 344.48 | | Ion Torrent | LQ | 15 | 12 | 5,787,993 | 3.9 | 89.1 | 98.4 |
| | | miRmix | | | | | proton | | | | | | | |
| | 10D | LT- | 50 | fmol | 344.48 | | Ion Torrent | LQ − | 15 | 12 | 3,302,266 | 4.9 | 89.0 | 98.0 |
| | | miRmix | | | | | proton | biotin | | | | | | |
| | | | Estimated | | | | | | | | | | | Total no. |
| | | | miRNA | | Plasma | | | | | | | | | of miRNAs |
| | | | concentration | | equivalents | | | | | | | | | represented |
| B: | 6A | | 100 | amol | 10 | μl | Ion Torrent | LQ | 20 | 12 | 338,867 | 5.4 | 77.1 | 184 |
| total | | | | | | | PGM | | | | | | | |
| RNA from | 6B | | 100 | amol | 10 | μl | Ion Torrent | LQ | 22 | 12 | 337,375 | 13.0 | 79.5 | 136 |
| human | | | | | | | PGM | | | | | | | |
| blood | 6C | | 100 | amol | 10 | μl | Ion Torrent | LQ | 20 | 12 | 313,864 | 8.3 | 76.8 | 196 |
| plasma | | | | | | | PGM | | | | | | | |
| | 6D | | 500 | amol | 51 | μl | Ion Torrent | LQ | 20 | 12 | 296,922 | 3.7 | 86.9 | 223 |
| | | | | | | | PGM | | | | | | | |
| | 7A | | 90 | amol | 110 | μl | Ion Torrent | LQ | 20 | 10 | 4,754,997 | 2.2 | 96.4 | 127 |
| | | | | | | | proton | | | | | | | |
| | 7B | | 125 | amol | 110 | μl | Ion Torrent | LQ | 20 | 10 | 3,562,371 | 2.7 | 95.6 | 137 |
| | | | | | | | proton | | | | | | | |
| | 8A | | 100 | amol | 10 | μl | Ion Torrent | LQ | 20 | 12 | 197,598 | 3.4 | 68.9 | 211 |
| | | | | | | | PGM | | | | | | | |
| | 8B | | 250 | amol | 25 | μl | Ion Torrent | LQ | 20 | 12 | 191,701 | 2.7 | 63.3 | 223 |
| | | | | | | | PGM | | | | | | | |
| | 8C | | 500 | amol | 51 | μl | Ion Torrent | LQ | 20 | 12 | 533,535 | 2.1 | 85.5 | 221 |
| | | | | | | | PGM | | | | | | | |
| | 8D | | 750 | amol | 77 | μl | Ion Torrent | LQ | 20 | 12 | 635,374 | 1.9 | 88.5 | 209 |
| | | | | | | | PGM | | | | | | | |
| | 8E | | 1000 | amol | 102 | μl | Ion Torrent | LQ | 20 | 12 | 316,475 | 2.8 | 78.2 | 202 |
| | | | | | | | PGM | | | | | | | |

Libraries generated from the synthetic miRNA mixtures or total RNA isolated from human blood plasma. Each library is assigned a number designation associated with individual cloning experiments and, where applicable, a letter when samples were multiplexed. These designations are used throughout the text and figure legends to refer to specific libraries analyzed. The LQ and 2-linker methods are distinguished by black and red text, respectively, in subsequent figures.
Synthetic mixture, RNA concentration, RNA quantity, sequencing platform, cloning method used and total read number obtained are as indicated. LQ + heat refers to the library generated with a 65° C. heat step prior to the RT reaction. LQ − biotin refers to the library generated using the LQ method in the absence of biotin. RNA quantity for the miRNA mixture was calculated based on molecular weight of 21 nt ssRNA and by using a standard curve and single Taqman assay for miRNA from human blood plasma (see Materials and Methods). Total read number includes all reads ≧17 nt and that contained adapter and randomer (NXG) sequences. ‘% filtered reads’ is the percent of total reads removed by hotspot read filtering. The reference used for mapping the synthetic miRNA mixture was the appropriate reference set generated as explained in the Materials and Methods section and was the human genome for libraries made from total RNA from human blood plasma. ‘% reads mapped’ is the percentage of remaining reads mapped to the appropriate reference set. Reads with more than 10 reads per million were used to determine the ‘% reference coverage’ for the miRNA mixture libraries and to determine the ‘total no. of miRNAs represented’ for plasma RNA.

TaqMan Array Microfluidic Card microRNA Profile

Quantification of and profiling miRNA content in RNA from plasma was performed using the TaqMan Array Human MicroRNA A card Set v3.0 together with the Megaplex™ RT primers, Human Pool A v3.0 (Life Technologies) following the protocol for profiling with pre-amplification using Megaplex™ PreAmp primers, Human Pool A v2.1.

RNA was reverse transcribed in a 5.0 μl reaction using the Megaplex primer pool A or B. Briefly, 2.0 μl total RNA was mixed with 1× reaction buffer, 3 mM MgCl2, 2 units RNase Inhibitor, 2.7 mM dNTPs, 1× Megaplex Primer pool A or B, and 75 units MultiScribe Reverse Transcriptase. Reverse transcription reaction conditions were: 40× (16° C. 2 min., 42° C. 1 min., 50° C. 1 sec.), 85° C. 5 min.

Preamplification was performed in a 5.0 μl reaction by mixing 0.5 μl cDNA with 1× MegaPlex PreAmp Primers pool A or B and 1× PreAmp master mix (Life Technologies). Preamplification reaction conditions were: 95° C. 10 min., 55° C. 2 min., 72° C. 2 min., 12× (95° C. 15 sec., 60° C. 4 min.), 99° C. 10 min.

Preamplification product was diluted 1:4 in TE and 8.0 μl was mixed with 400 μl 2× TaqMan Universal PCR Master Mix No AmpErase UNG (Life Technologies) and 392 μl RNase free H2O. 90.0 ul of this mix was loaded into each port of the Microfluidic Array card according to the manufacturer's instructions. The Array card was run on a 7900 HT Real-Time instrument (Life Technologies) with the following reaction conditions: 50° C. 2 min., 95° C. 10 min., 40× (95° C. 15 sec., 60° C. 1 min.).

Quantification of miRNA in Total RNA from Human Blood Plasma: Use of miR-223

miRNA quantification from total RNA isolated from human blood plasma was assayed by TaqMan Low Density Array (TLDA) cards. Ct values (logarithmic scale) were converted to a linear scale reflective of molecule number, where Ct of 32=1 molecule. An abundant miRNA (miR-223) was used as a benchmark and determined to account for approximately 20% of all miRNA in the sample. The concentration of miR-223 in total RNA isolated from human blood plasma was determined by generating a standard curve using miR-223 and the LT-miRmix (containing approximately 1,000 miRNAs). The concentration of miR-223 was multiplied by 5 to determine the approximate concentration of miRNA in total RNA isolated from human blood plasma.

Generation of Minus-Biotin Libraries

Minus-biotin libraries were generated as described in Materials and Methods with the following changes: size-selected circularized cDNA was eluted in the absence of streptavidin beads, ethanol precipitated, resuspended in 10 μl of RNA milliQ H2O, and 5 μl was used as template for first round PCR amplification as described.

Results

Key Features of the Method

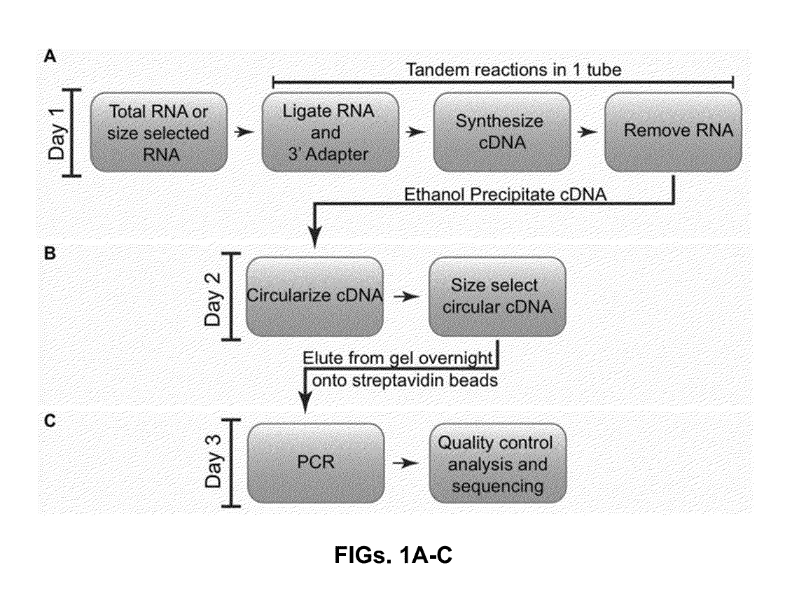

Overview To establish a method for preparing cDNA libraries for high-throughput sequencing from a very low quantity (LQ) of input RNA, a relatively straightforward and streamlined cloning protocol that minimizes sample loss by reducing the number of sample extraction and gel purification steps compared to conventional cloning protocols while enabling cloning from significantly less material compared to commercially available streamlined methods. We also incorporated provisions for sample multiplexing and future development of high-throughput applications. The protocol involved sequential linker ligation and RT reactions in a single tube (FIG. 1A), where a single adapter is ligated to the 3′ end of the RNA, followed by generation of biotin-containing reverse transcribed cDNAs. cDNAs were then circularized, the biotin-containing cDNAs are isolated (FIG. 1B) and libraries were amplified by PCR (FIG. 1C). This streamlined method allowed for preparation of libraries ready for quantification and sequencing in 2-3 days (FIG. 1). The steps of an exemplary protocol are diagrammed in FIG. 2 and described in detail in the Materials and Methods section of this Example. Specific features of the protocol that contribute to its enhanced sensitivity and simplicity are presented below.

### Single-Tube, Sequential Ligation and Reverse Transcription Reactions

Ligation reactions were performed with truncated and mutated T4 RNA Ligase 2 (T4Rnl2tr K227Q) at 30° C. for 6 h. T4Rnl2tr K227Q carries out a more specific and efficient ligation between RNA and 3′ adapter compared to wild-type T4Rn12 and the 30° C. ligation reaction temperature reduces sequence biases that may be introduced by RNA secondary structure such as stem-loops (33,34).

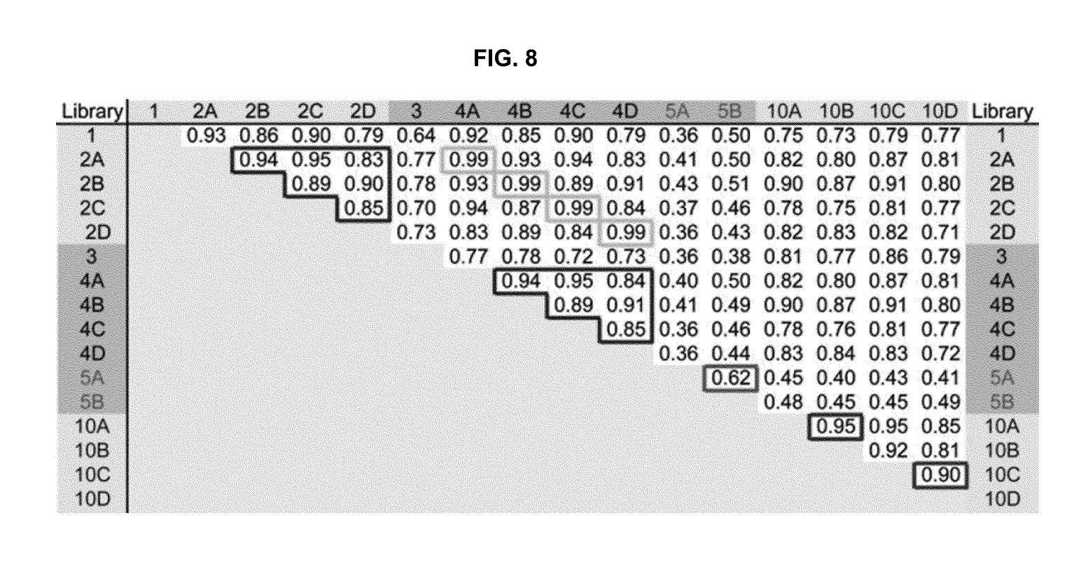

To promote efficient first-strand cDNA synthesis, the RT primer oligonucleotide was pre-annealed to the 3′ adapter (adapter::RT oligo; FIG. 2A) prior to the ligation reaction. This provided for maintaining equimolar stoichiometry of the adapter and RT oligo throughout the ligation and, importantly, pairs ligated RNA with a barcoded RT oligo. Moreover, since no extraction or precipitation steps occur after ligation, the pre-annealed RT primer serves as a substrate for RT of ligated RNA in sequential single-tube ligation and RT reactions. Accordingly, immediately following ligation, the thermostable reverse transcriptase SuperScript III (Invitrogen), RT buffer and reaction components were added directly to the ligation mixture. This direct transition from the ligation to the RT reaction avoids a gel purification step necessary in some other methods and hence reduces sample losses. Finally, by generating cDNAs at 45° C., efficient extension was achieved while maintaining annealing of the 3′ adapter::RT oligo hybrid. Importantly, removal of a denaturing step prior to the RT reaction did not inhibit the ability to isolate sequences with high GC content and libraries generated with (10B) or without (10A) a 65° C. heat step for 5 min prior to the RT correlated very well (FIG. 8).

Novel Features Introduced into cDNAs

Notable features of the RT oligo include a 5′ terminal Guanine (G) to minimize nucleotide bias inherent to the circular ligase, a barcode to enable sample multiplexing, a randomer to identify PCR hotspots (see Materials and Methods) and internal deoxyuracil (dUTP) nucleotides to enable linearization of circular cDNAs by Uracil-D-glycosylase (UDG, aka Uracil-N glycosylase, UNG) in the first round of PCR (Table 1 and FIG. 2). Finally, to promote efficient recovery of purified cDNA (FIG. 2A), biotinylated dCTP and biotinylated dATP are included in the cDNA reaction (FIG. 2B and Materials and Methods section). Importantly, biotinylated nucleotides are required for recovery of cDNAs resulting from successful ligation reactions. In the absence of biotin, only cDNAs reflective of RT oligos are recovered as evidenced by short (44-49 nt) first round PCR products.

Circularization and Isolation of Biotinylated cDNA

Following first-strand cDNA synthesis, RNA was removed by base hydrolysis and cDNAs are ethanol precipitated with 10M ammonium acetate at room temperature, facilitating removal of free 3′ adapter and unincorporated nucleotides (35,36) (FIG. 2C). All recovered material was circularized using CircLigase II (Epicenter) (FIG. 2D) and fractionated on a 10% denaturing gel to obtain circular single-stranded DNA of the desired length: 69-74 nucleotides for cloned small RNAs. This was the only gel purification step in the protocol prior to sample amplification, hence this step allowed for size selection of circularized ‘+ insert’ products and also served as a first step in separation of ‘+ insert’ material away from ‘no insert’ material. Excised ‘+ insert’ cDNAs were eluted from the gel overnight in the presence of streptavidin beads, allowing for selective binding and hence a full isolation of biotin containing ‘+ insert’ cDNAs away from remaining ‘no insert’ (non-biotinylated) material. Beads were washed and the streptavidin bead-bound ‘+ insert’ cDNAs are amplified directly from the beads in the first round PCR.

Utilization of Deoxyuracil in PCR Amplification of Libraries

In the first round of PCR, primers complementary to sequences flanking the cloned sequence (Table 2, FIGS. 2E and F) and Taqman Gene Expression Master Mix (Life Technologies) were added directly to streptavidin bead-bound ‘+ insert’ material. UNG in the master mix enables excision of dUTP from template molecules, promoting strand scission and generation of a linear PCR template. The master mix also contained a blend of dTTP/dUTP nucleotides: incorporation of dUTP into new amplicons serves to minimize carry-over PCR contamination between first round products. First round PCR products were resolved on an 8% non-denaturing polyacrylamide gel and appropriate 65-70 nt products are excised. Primers complementary to the 5′ and 3′ sequences of first round PCR products that included adapters specific for sequencing on either the Ion Torrent or Illumina platform (FIG. 2G and Table 2) were then used in the second round of PCR for final library amplification prior to sequencing.

Computational Analysis of cDNA Libraries

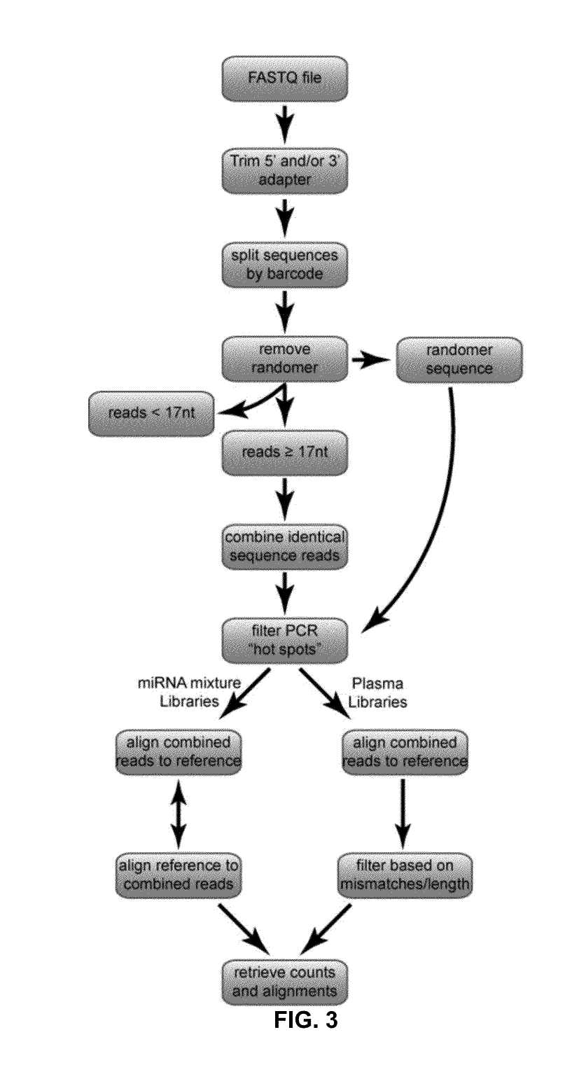

In order to identify cloned sequences, an in-house computational pipeline was used (FIG. 3 and Materials and Methods section). In this pipeline, first, 5′ and 3′ adapter sequences (Ion Torrent-derived libraries) or 3′ adapter sequences (Illumina-derived libraries) were removed. Second, by using the unique 3-6 nucleotide (nt) barcode sequence incorporated into each library, multiplexed samples were split by barcode. Next, the randomer sequences were removed and saved for later PCR hotspot analysis. Reads were then filtered for length. All sequences shorter than 17 nt were discarded, as this limited read length could not be mapped with high confidence. All remaining ≧17 nt reads having an identical sequence were combined and counted. Identical reads were checked for PCR hotspots by comparing the distribution of the randomers associated with each sequence to the expected distribution of randomers (determined from the sequencing data; see Materials and Methods section) and filtered accordingly. Remaining reads were aligned to the reference sequences using the Bowtie alignment tool (28) followed by a final filtering of reads based on length and mismatch cut-offs as described in Materials and Methods section.

Method Evaluation and Application

An ideal method for generating cDNA libraries from small RNAs should be sensitive, reproducible over a wide range of input RNA quantity and should accurately represent the sequence profile of the input RNA. Therefore, rigorous assessments of these criteria were performed by first creating libraries from the Life Technologies synthetic miRNA mixture (LT-miRmix) using both our LQ method and the established 2-linker cloning method (13). While the LT-miRmix was complex and better represented sequences that may be encountered in biological samples, limited sequence annotation and unknown molarity for the contents of the LT-miRmix led to further examination of the LQ method using a well-characterized miRNA mixture (29-miRmix) comprised of 29 synthetic miRNAs with known stoichiometry and sequence context (23). The potential applicability of the method was also assessed in a clinical setting by cloning cDNA libraries from small quantities of total RNA isolated from human blood plasma.

Libraries generated using the synthetic miRNA mixtures were used to assess the accuracy of the method in recovering sequences in a standardized set of RNA. Sequences obtained from cDNA libraries generated from the mixtures were mapped to the LT-miRNA mixture reference set of sequences (Materials and Methods) or the set of 29-miRNAs. The accuracy of the method was then assessed in terms of read length distribution, 5′ end identity and heterogeneity (particularly from potential terminal transferase activity of the RT enzyme), 3′ end identity and heterogeneity, and potential sequence bias or mutagenesis introduced through the use of biotinylated nucleotides. Central to this assessment was a comparison of libraries generated using the LQ method with and without biotin as well as to those made with the biotin-free 2-linker approach.

cDNA Library Sequence Overview

Libraries were generated from two synthetic miRNA mixtures, one containing approximately 1,000 miRNA sequences provided by Life Technologies and a second containing 29 miRNAs (23), as well as total RNA isolated from normal human blood plasma. The average number of reads obtained from libraries sequenced on the Ion Torrent and Illumina platforms ranged from 190,000 to >1,000,000 depending on the number of samples multiplexed and the platform used (Table 3).

As RNA input quantity decreased, the number of PCR cycles required to yield detectable PCR product increased (Table 3). In general, increasing the number of PCR cycles affected the percentage of hotspots detected in our libraries, as evidenced by clusters of identical reads containing an identical randomer sequence. The libraries that required relatively more PCR amplification steps could contain up to 14% of total reads associated with hotspot amplification (Table 3). These results emphasized the importance of including randomer sequence tags in the cloning oligo backbone for the identification and compression of hotspot reads. The impact of using a 6 versus a 4 nt randomer was also examined; no particular advantage to using a longer randomer was observed.

After compression of hotspot sequence reads, remaining reads were mapped to the reference sequence set corresponding to the source RNA: the synthetic mixture reference set for LT-miRNAmix libraries, the 29 miRNA sequences for 29-miRmix libraries, or the small RNA reference and the human genome for plasma-derived libraries (Materials and Methods section). The percentage of sequences that mapped to the respective reference sequences varied from 51% to 96%, somewhat dependent upon the quantity of input RNA. For example, in libraries generated from the synthetic miRNA mixtures, greater quantities of input RNA tended to result in libraries with higher percent mapping to the reference. However, for libraries generated from lower amounts of input RNA, the reference sequence coverage was still substantial (at least 93%) (Table 3).

For libraries generated from human blood plasma RNA, we identified sequences corresponding to a broad repertoire of small RNAs matching the human genome. The majority of sequences mapped to human miRNAs, while the remainder mapped to ribosomal RNA (rRNA), transfer RNA (tRNA) fragments, other circulating RNAs such as Y RNA (37), and also sequences annotated as Piwi-interacting RNAs (piRNAs) (38,39). Future study is required to assess the potential biological relevance of these various circulating RNA populations.

For libraries made from synthetic RNA input or from plasma RNA input, a fraction of reads (from 49% down to 4%) did not map to the respective reference sequence set. Prominent among these non-mapping sequences were apparent plasmid vector sequences, indicating that small amounts of laboratory nucleic acid contamination from unrelated experiments can enter the workflow.

Read Length Distribution

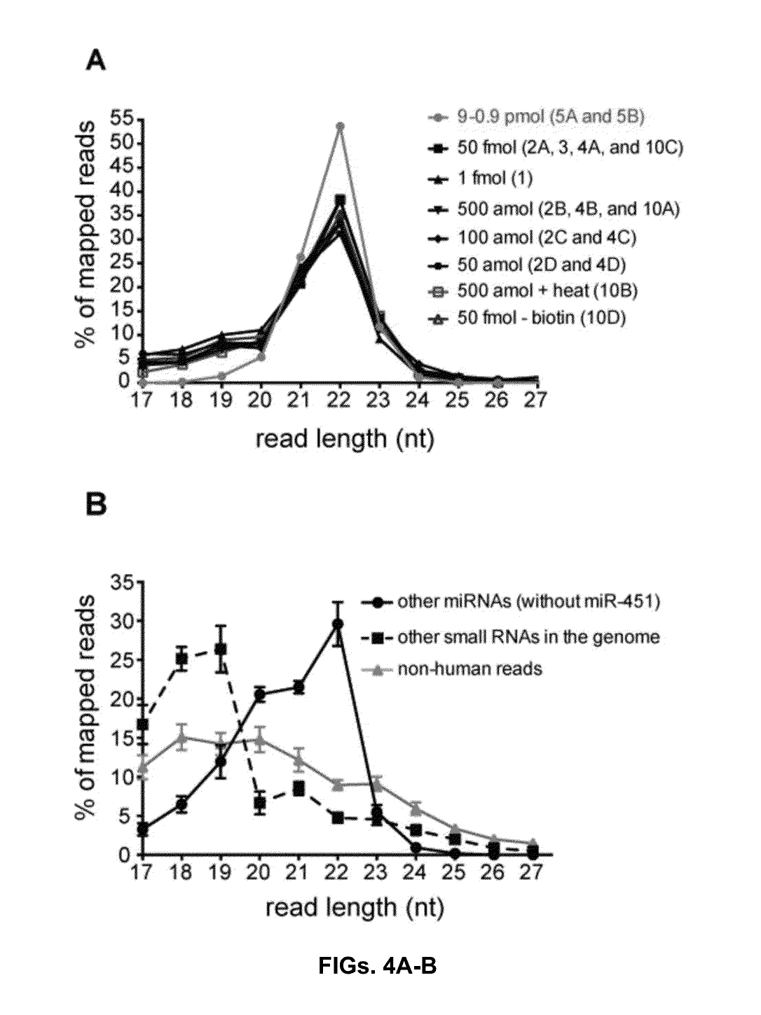

The expected length distribution of cDNA sequences obtained by the LQ method should depend upon the length of input material and on the size of circular ligated material excised from the gel. This analysis sought to clone small RNAs in the range of 18-24 nt from either synthetic miRNA mixtures or from total RNA isolated from human blood plasma. The length distribution of RNAs cloned from synthetic mixtures using the LQ method was consistent regardless of input RNA concentration, the addition to heat prior to the RT step, or the absence of biotin. Interestingly, the cDNA length distribution of the LQ method included a 17-20 nt fraction that was significantly reduced in the length distribution isolated from the 2-linker method (FIG. 4A), perhaps owing to the additional size-specific gel purification step included in the 2-linker method.

To assess length distribution of RNAs cloned from total plasma RNA, we divided reads into three groups: (i) human miRNAs (excluding the highly abundant miR-451a; see below for explanation), (ii) other small RNAs mapping to the human genome and (iii) reads that did not match the human genome. For miRNAs, the majority of reads (72%) ranged in length from 20 to 22 nt. Similar to what was observed with the miRNA mixtures, shorter reads (22%) included small RNA fragments that map to miRNAs and reflect mainly truncation at the 3′ ends, and the remaining reads were sequences longer than 22 nt. Moreover, the size distribution for these sequences, as well as other human and non-human small RNAs, fell within the size range expected from the size selection step of circularized cDNA in our protocol (FIGS. 2 and 4B; Materials and Methods section).

5′ and 3′ End Nucleotide Bias

In order to assess potential nucleotide-generated bias in either the ligation of RNA to the 3′ adapter or in the circular ligation of generated cDNAs, the 5′ and 3′ nucleotides of sequences cloned from the sequence-diverse LT-miRNA mixture were examined. Overall, a relative increase in 5′ Adenine (A) and a decrease in 5′ Cytosine (C) was observed in the LQ method versus the 2-linker method (FIG. 5A). This bias may be attributed to the use of different enzymes in the 5′ end ligation reactions. In the LQ method, CircLigase II is used to ligate the 3′ end of the cDNA (corresponding to the 5′ nt of the RNA) to the 5′ end of the RT primer, whereas the 2-linker method utilizes T4 RNA ligase I to ligate the 5′ end of RNA to a 5′ end adapter. T4 RNA ligase I exhibits sequence dependent ligation preferences (33) that very likely differ from those of CircLigase II. Interestingly, the CircLigase II enzyme exhibits a preference for ligation of 3′ Thymine (T) to 5′ Guanine (G) (Epicentre personal communication). The LQ RT oligo contained a 5′ G, and so this intrinsic T-to-G ligation bias for CircLigase II could explain the observed enrichment in LQ libraries for cDNA sequences corresponding to RNAs containing a 5′ A (FIG. 5A). Additionally, the 2-linker method employed 5′ end adapters with different nucleotides at the 3′ end (Table 1). Observed sequence bias among the two libraries generated by the 2-linker method is likely due to the sequence variation at the 3′ end (33,40,41). In contrast, the 3′ end ligations in both methods utilized the same enzyme, truncated and mutated T4 RNA ligase 2 (T4 Rnl2tr K227Q). Therefore, no 3′ nucleotide (3′ nt) bias was expected in libraries generated from the two different methods, and indeed no significant difference was observed (FIG. 5B).

Effect of Biotin Incorporation on Sequence Accuracy

To the best of the present inventors' knowledge, this is the first use of biotinylated nucleotide incorporation into cDNAs generated from linker-ligated RNA. Moreover, at the outset, it was not clear how competent SuperScript III (Invitrogen) would be at incorporating biotinylated nucleotides or whether the fidelity of Amplitaq Gold DNA Polymerase (Invitrogen) would be impacted by the presence of biotinylated nucleotides in the cDNA template. To determine whether incorporation of biotinylated A and biotinylated C could lead to preferential isolation of RNAs with a high Guanine (G) and/or Uracil (U) content, and/or whether biotinylated nucleotides would be mutagenic in this context, the G/U content of mapped reads from the 2-linker method was compared to the mapped reads from the LQ method and found no significant difference between the two populations (FIG. 5C). As a more direct comparison, using the LQ method, 50 fmol of LT-miRmix was cloned with and without biotin incorporation. Consistent with comparison of the LQ and 2-linker methods, there was no significant difference in the G/U content of sequences isolated in the presence or absence of biotin (FIG. 5C). Additionally, the relationship between read count (as measured by proportion of reads) and G/U content was examined. The G/U content did not directly correlate with read count when either high (2.5 pmol) or low (500 amol) amounts of 29-miRmix RNA was cloned. Together, these results indicate that incorporation of biotinylated A and C does not appreciably affect the base composition of cDNAs recovered in the biotin-containing LQ procedure. Note that the RT reaction contains a mixture of biotinylated and non-biotinylated dCTP and dATP in ratios of 0.54:1 and 0.43:1, respectively. These ratios were optimized for cloning of small RNA cDNAs; it is possible that the proportion of biotinylated nucleotides in the RT reaction could be adjusted for applications involving the generation of appreciably longer cDNAs.

To assess possible mutagenic effects of biotinylated nucleotides, the LT-miRmix subset reference (Materials and Methods section) was used to compare any apparent mutation rates at G and U positions among sequences generated from each cloning method as well as from the LQ method with and without biotin incorporation. There were no significant differences in nucleotide representation, indicating that the incorporation of biotinylated nucleotides was not appreciably mutagenic (FIG. 5D).

5′ Additions, 3′ Variations and miRNA Isoforms

The LQ method involves generation of cDNAs without a 5′ linker, offering the advantage of cloning material independent of the presence of a 5′ monophosphate structure. However, absence of a linker sequence at the 5′ end of the template RNA introduces the potential for confounding effects of terminal transferase (TdT) activity of the reverse transcriptase. Although Superscript III reverse transcriptase has minimal TdT activity, we nevertheless identified significant 5′ nt additions during the early stages of method development (libraries D1 and D2, FIG. 6A). These 5′ additions were reduced from >22% (in libraries D1 and D2) to <3% (in libraries 1-4D) by reducing the quantity of RT enzyme to 0.5 units in each reaction, and by shortening the reaction time to 5 min (FIG. 6A).

Because miRNA 3′ end heterogeneity has been characterized in biological samples (42,43), it was sought to verify the ability of our LQ cloning method to identify these sequence variations. To do so, a profile of sequences with length variability in the LT-miRmix subset reference was generated as described in the Materials and Methods section. Sequence length profiles identified in the LQ versus 2-linker method indicate a similar ability for both methods to clone a wide range of miRNAs including those with 3′ end heterogeneity (FIG. 6B). Due to the 5′ independent nature of the protocol, it was not surprising that miRNAs with 5′ end truncations were isolated from both the synthetic miRNA mixtures analyzed (data not shown). It is likely that these truncated molecules are due to heterogeneity arising at oligo synthesis or are generated by spurious base hydrolysis. Importantly, these sequence variations were missed by cloning methods employing a 5′ linker ligation that is dependent upon a 5′ phosphate and their identification highlights the importance of analyzing replicate libraries and employing differential analysis of the same method.

Interestingly, when plasma-derived miRNAs were compared to miRBase sequence annotations a number of 5′ and 3′ differences were identified between the annotated sequences and those obtained in our libraries. Such apparent miRNA isoforms (isomiRs) could reflect alternative processing of miRNA transcripts or incorrect sequence annotation. IsomiRs are of interest, since alterations in the 5′ end of a miRNA would change the seed sequence, and therefore alter target recognition, while 3′ end modifications may affect miRNA stability and/or function (44-46).

Sensitivity and Reproducibility

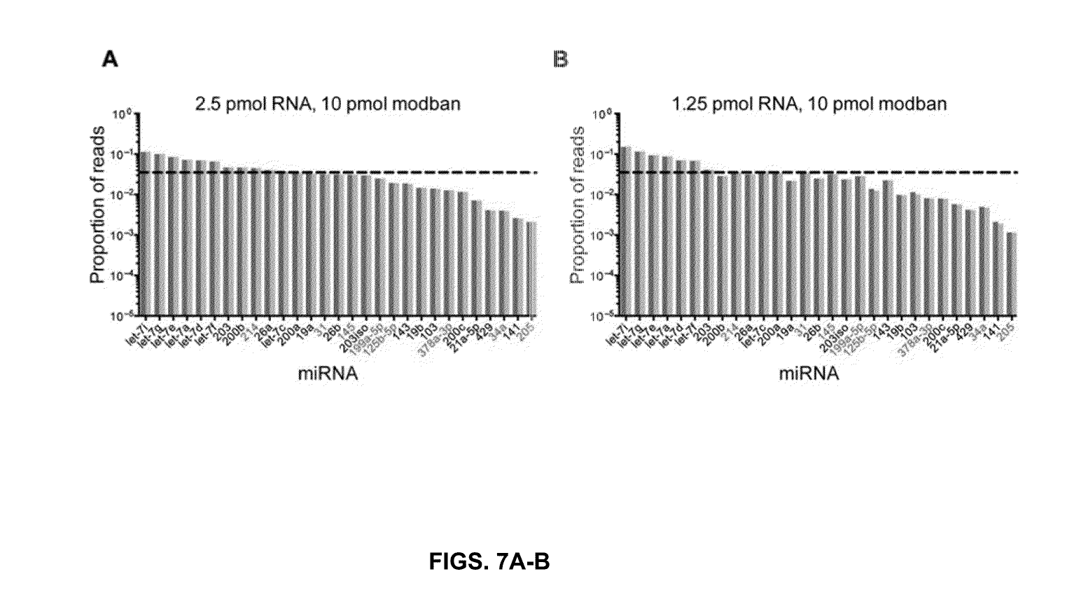

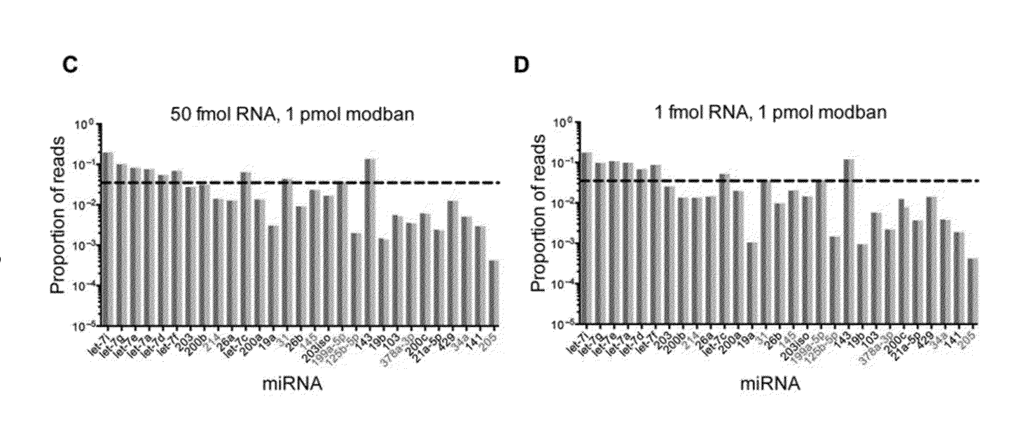

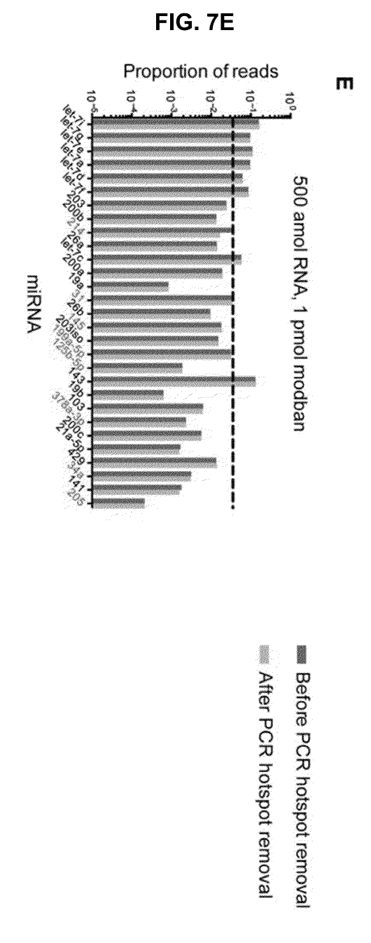

To test method sensitivity, libraries were generated across 3-4 orders of magnitudes using the synthetic miRNA mixtures (from 50 amol or 500 amol to 50 fmol) and across two orders of magnitude from human blood plasma (10-110 plasma equivalents corresponding to ˜90 amol-1,000 amol miRNA). From the LT-miRNA mixture, using the LQ method >93% of the reference miRNA sequences and >91% miRNA reference sequences were recovered coverage using the 2-linker method. In order to rigorously examine the ability to clone miRNAs in a sequence independent manner, libraries generated from decreasing input concentrations of the 29-miRmix were analyzed. Importantly, 100% of miRNA sequences in the mixture were isolated (Table 3 and FIG. 7). Compared to methods developed by Zhang et al. (23) and Heyer et al. (personal communication), we observed underrepresentation of some sequences using our method. This apparent sequence bias became more pronounced with lower input concentrations. However, we were unable to identify 5′ end, 3′ end or general sequence context features predictive of this underrepresentation. It is possible that incorporation of additional modifications to the LQ method may enhance uniformity of sequence recovery. Possible modifications could include: heating RNA samples prior to 3′ adapter ligation, the use of PEG-8000 in ligation reactions, a degenerate 5′ end of the RT oligo and further optimization of enzymatic steps including buffer compositions (23,33,41,47-49). Additionally, it is important to note that use of the LQ method enables generation of libraries from significantly less material than these other methods as well as those commercially available.

Cloning from human blood plasma demonstrated recovery of a range of 127-223 mature miRNA sequences that had >10 reads per million for those reads that mapped the human genome. This range was dependent upon the depth of the library examined and the extent of hemolysis that could be indicated by miR-451a representation (see below for explanation of miR-451a) (Table 3). Together, these data demonstrate significant sequence coverage and identification using this LQ cloning method.

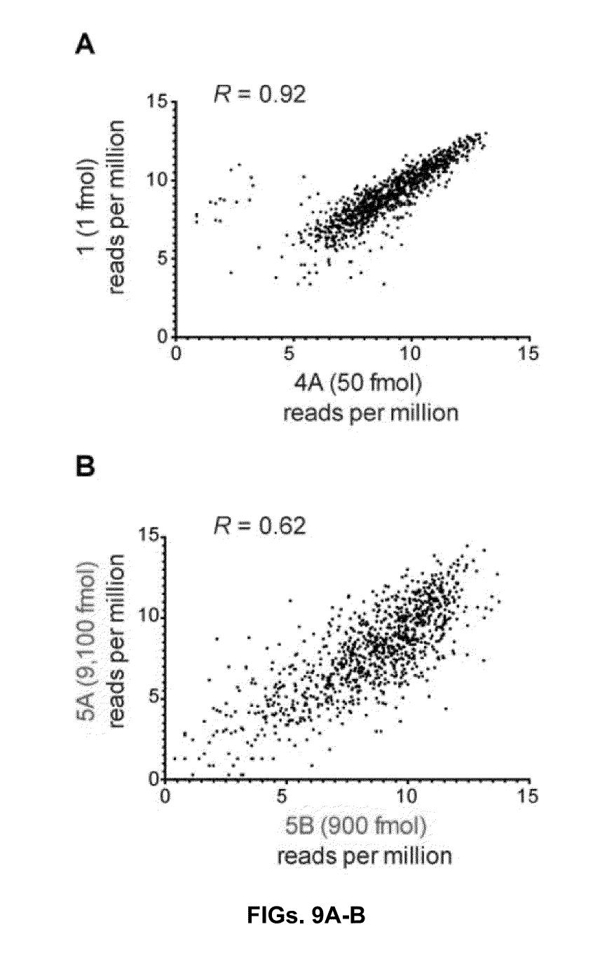

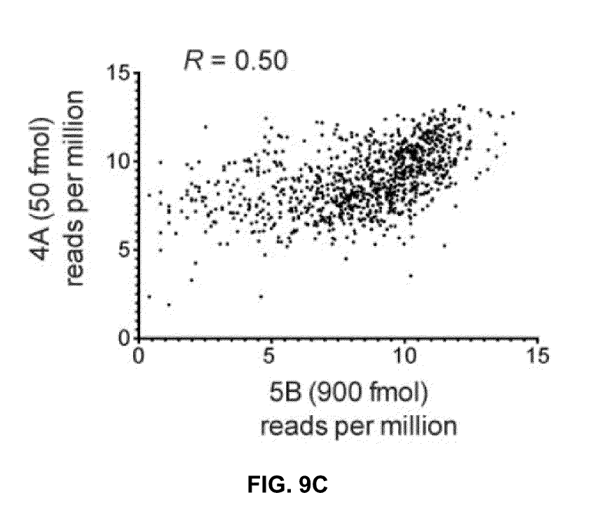

To assess the consistency of sequences recovered by the LQ approach, the correlation of sequence read counts was examined among libraries from the same source RNA. Read counts from synthetic miRNA-derived libraries correlated well whether from the same amount or varying amounts of input RNA (FIG. 8, dark blue boxes). Comparison of reads from two libraries generated from the LQ method demonstrates good correlation (FIG. 9A). Not unexpectedly, there were some non-correlating outliers for certain low abundance sequences. Generally, libraries generated from the LQ method were more highly correlated (FIG. 8, dark blue boxes) than those generated using the 2-linker method (FIG. 8, red box, and FIG. 9B). Finally, while libraries generated by the LQ method and 2-linker method had similar read coverage, read counts for individual sequences were not well correlated between the two methods (Table 3 and FIG. 9C).

To assess reproducibility between sequencing platforms, a single library was generated and then amplified with either Ion Torrent (Libraries 2A-2D) or Illumina (Libraries 4A-4D) specific adapters at the second round of PCR. Sequencing on the Ion Torrent or Illumina platform yielded similar percentages of reference sequence covered (Table 3) and high correlation of sequence read counts in corresponding libraries (generated from the same input material) (FIG. 8, light blue boxes).

Analysis of miRNA read counts isolated from human blood plasma showed correlation above 0.98 for all library comparisons. This substantiates the view that this LQ method is sufficiently sensitive and reproducible for application in the context of scarce clinical samples containing very low quantities of RNA.

miRNA Repertoire of Human Blood Plasma

Identification of miRNAs in blood plasma (4) has led to the characterization and profiling of circulating miRNA populations in a variety of disease contexts where the miRNA expression has been shown to indicate tissue damage and disease status (for examples see 2, 50-53). A confounding issue to the circulating miRNA profile is the presence of blood-cell-associated miRNAs (54). In particular, the miRNA repertoire detected in plasma RNA is highly sensitive to the degree of hemolysis that can occur during sample isolation and processing (54, 55). Remarkably, even traces (0.031% (v/v)) of red blood cells in plasma can alter the miRNA expression profile compared to non-hemolyzed samples (56).

Examination of cDNA libraries prepared from human blood plasma identified a range (127-223) of significantly expressed mature miRNAs. Analysis of miRNA read counts done on all cDNA libraries revealed significant representation of blood-cell-independent miRNAs as well as a number of miRNAs that have not been carefully studied in the context of hemolysis (Table 4). Consistent with previous findings, hemolysis-associated miRNAs were also identified. For example, miR-451a accounted for 58-82% of all reads that map to the human genome (FIG. 10), indicating that these blood plasma samples had suffered significant hemolysis. Because miR-451a reads constituted a predominant and variable fraction of our libraries, miR-451a reads were excluded for certain aspects of the analysis of the other miRNAs represented in the libraries.

TABLE 4
Top-40 miRNAs isolated from human blood plasma

| miRNA | Average reads per million | Hemolysis associated |
|---|---|---|
| hsa-miR-451a | 685,118 | yes^{56,55,65,54} |
| hsa-miR-144-3p | 33,490 | yes⁶⁵ |
| hsa-miR-16-5p | 26,367 | yes^{64,56,55,54} |
| hsa-miR-15a-5p | 10,525 | |
| hsa-miR-22-3p | 6,994 | |
| hsa-miR-20a-5p | 4,833 | yes⁵⁵ |
| hsa-miR-142-3p | 3,852 | no⁵⁵ |
| hsa-let-7g-5p | 3,387 | |
| hsa-miR-29c-3p | 3,326 | |
| hsa-miR-486-5p | 3,267 | yes⁵⁴ |
| hsa-miR-223-3p | 2,950 | no^{64,56,55,65,54} |
| hsa-miR-93-5p | 2,561 | no⁵⁵ |
| hsa-miR-15b-5p | 2,330 | yes^{64,56} |
| hsa-miR-103a-3p | 2,301 | yes⁵⁵ |
| hsa-miR-21-5p | 2,264 | yes⁵⁵ |
| hsa-let-7i-5p | 2,191 | |
| hsa-miR-26a-5p | 1,979 | |
| hsa-miR-101-3p | 1,915 | |
| hsa-miR-107 | 1,911 | |
| hsa-miR-92a-3p | 1,682 | yes^{56,54} |
| hsa-miR-142-5p | 1,568 | no⁵⁵ |
| hsa-miR-126-3p | 1,448 | no⁵⁵ |
| hsa-miR-23a-3p | 1,348 | no⁶³ |
| hsa-miR-29a-3p | 1,275 | no⁵⁵ |
| hsa-miR-30e-5p | 1,261 | |
| hsa-miR-27a-3p | 1,215 | |
| hsa-miR-185-5p | 1,156 | |
| hsa-let-7a-5p | 1,089 | no⁵⁴ |
| hsa-miR-29b-3p | 1,001 | |
| hsa-miR-26b-5p | 943 | |
| hsa-miR-25-3p | 928 | |
| hsa-let-7b-5p | 902 | yes⁵⁵ |
| hsa-miR-19b-3p | 870 | |
| hsa-miR-122-5p | 852 | no^{64,54,66}, yes⁵⁵ |
| hsa-miR-425-5p | 767 | yes⁵⁵ |
| hsa-miR-32-5p | 757 | |
| hsa-miR-18a-5p | 722 | |
| hsa-let-7f-5p | 701 | |
| hsa-miR-24-3p | 658 | no⁵⁵, slightly⁶⁴ |
| hsa-miR-146a-5p | 512 | no⁵⁵ |

The average (in reads per million) of indicated mature miRNAs in libraries 6A-8E.
Hemolysis association, where clearly identified, is referred to and reference(s) are indicated.

### REFERENCES

1. Bartel, MicroRNAs: genomics, biogenesis, mechanism, and function. Cell 2004; 116:281-297.
2. Mitchell et al., Circulating microRNAs as stable blood-based markers for cancer detection. Proc. Natl. Acad. Sci. U.S.A. 2008; 105:10513-10518.
3. Lawrie et al., Detection of elevated levels of tumour-associated microRNAs in serum of patients with diffuse large B-cell lymphoma. Br. J. Haematol. 2008; 141:672-675.
4. Chim et al., Detection and characterization of placental microRNAs in maternal plasma. Clin. Chem. 2008; 54:482-490.
5. Chen et al., Characterization of microRNAs in serum: a novel class of biomarkers for diagnosis of cancer and other diseases. Cell Res. 2008; 18:997-1006.
6. Weber et al., The microRNA spectrum in 12 body fluids. Clin. Chem. 2010; 56:1733-1741.
7. Lu et al., MicroRNA expression profiles classify human cancers. Nature 2005; 435:834-838.
8. He et al., A microRNA polycistron as a potential human oncogene. Nature 2005; 435:828-833.
9. Taylor and Gercel-Taylor, MicroRNA signatures of tumor-derived exosomes as diagnostic biomarkers of ovarian cancer. Gynecol. Oncol. 2008; 110:13-21.
10. Pfeffer et al., Cloning of small RNA molecules. Curr. Protoc. Mol. Biol. 2005. Chapter 26, Unit 26.4.
11. König et al., iCLIP reveals the function of hnRNP particles in splicing at individual nucleotide resolution. Nat. Struct. Mol. Biol. 2010; 17:909-915.
12. Morin et al., Application of massively parallel sequencing to microRNA profiling and discovery in human embryonic stem cells. Genome Res. 2008; 18:610-621.
13. Gu et al., CapSeq and CIP-TAP identify Pol II start sites and reveal capped small RNAs as C. elegans piRNA precursors. Cell 2012; 151:1488-1500.
14. Williams et al., Comprehensive profiling of circulating microRNA via small RNA sequencing of cDNA libraries reveals biomarker potential and limitations. Proc. Natl. Acad. Sci. U.S.A. 2013; 110:4255-4260.
15. Kwon, Small RNA library preparation for next-generation sequencing by single ligation, extension and circularization technology. Biotechnol. Lett. 2011; 33:1633-1641.
16. Mendell and Olson, MicroRNAs in stress signaling and human disease. Cell 2012; 148:1172-1187.
17. Farazi et al., MicroRNAs in human cancer. Adv. Exp. Med. Biol. 2013; 774:1-20.
18. Islam et al., Quantitative single-cell RNA-seq with unique molecular identifiers. Nat. Methods 2014; 11:163-166.
19. Adiconis et al., Comparative analysis of RNA sequencing methods for degraded or low-input samples. Nat. Methods 2013; 10:623-629.
20. Ramskold et al., Full-length mRNA-Seq from single-cell levels of RNA and individual circulating tumor cells. Nat. Biotechnol. 2012; 30:777-782.
21. Picelli et al., Full-length RNA-seq from single cells using Smart-seq2. Nat. Protoc. 2014; 9:171-181.
22. Picelli et al., Smart-seq2 for sensitive full-length transcriptome profiling in single cells. Nat. Methods 2013; 10:1096-1098.
23. Zhang et al., High-efficiency RNA cloning enables accurate quantification of miRNA expression by deep sequencing. Genome Biol. 2013; 14:R109.
24. Ho et al., Structure and mechanism of RNA ligase. Structure 2004; 12:327-339.
25. Ho and Shuman, Bacteriophage T4 RNA ligase 2 (gp24.1) exemplifies a family of RNA ligases found in all phylogenetic domains. Proc. Natl. Acad. Sci. U.S.A. 2002; 99:12709-12714.
26. Ingolia et al., Genome-wide analysis in vivo of translation with nucleotide resolution using ribosome profiling. Science 2009; 324:218-223.
27. Martin, Cutadapt removes adapter sequences from high-throughput sequencing reads. EMBnet.journal. 2011; 171:10-12.
28. Langmead et al., Ultrafast and memory-efficient alignment of short DNA sequences to the human genome. Genome Biol. 2009; 10:R25.
29. Karolchik et al., The UCSC Table Browser data retrieval tool. Nucleic Acids Res. 2004; 32:D493-D496.
30. Sai Lakshmi and Agrawal, piRNABank: a web resource on classified and clustered Piwi-interacting RNAs. Nucleic Acids Res. 2008; 36:D173-D177.
31. Kin et al., fRNAdb: a platform for mining/annotating functional RNA candidates from non-coding RNA sequences. Nucleic Acids Res. 2007; 35:D145-D148.
32. Griffiths-Jones et al., miRBase: tools for microRNA genomics. Nucleic Acids Res. 2008; 36:D154-D158.
33. Hafner et al., RNA-ligase-dependent biases in miRNA representation in deep-sequenced small RNA cDNA libraries. RNA 2011; 17:1697-1712.
34. Viollet et al., T4 RNA ligase 2 truncated active site mutants: improved tools for RNA analysis. BMC Biotechnol. 2011; 11:72-86.
35. Okayama and Berg, High-efficiency cloning of full-length cDNA. Mol. Cell. Biol. 1982; 2:161-170.
36. Crouse and Amorese, Ethanol precipitation: ammonium acetate as an alternative to sodium acetate. Focus 1996; 18:17-20.
37. Dhahbi et al., 5′-YRNA fragments derived by processing of transcripts from specific YRNA genes and pseudogenes are abundant in human serum and plasma. Physiol. Genomics 2013; 45:990-998.
38. Cheng J., et al., piRNA, the new non-coding RNA, is aberrantly expressed in human cancer cells. Clin. Chim. Acta 2011; 412:1621-1625.
39. Lu et al., Identification of piRNAs in Hela cells by massive parallel sequencing. BMB Rep. 2010; 43:635-641.
40. Alon et al., Barcoding bias in high-throughput multiplex sequencing of miRNA. Genome Res. 2011; 21:1506-1511.
41. Sun et al., A bias-reducing strategy in profiling small RNAs using Solexa. RNA 2011; 17:2256-2262.
42. Lee et al., Complexity of the microRNA repertoire revealed by next-generation sequencing. RNA 2010; 16:2170-2180.
43. Ameres and Zamore, Diversifying microRNA sequence and function. Nat. Rev. Mol. Cell Biol. 2013; 14:475-488.
44. Ebhardt et al., Naturally occurring variations in sequence length creates microRNA isoforms that differ in argonaute effector complex specificity. Silence 2010; 1:12-18.
45. Neilsen et al., IsomiRs—the overlooked repertoire in the dynamic microRNAome. Trends Genet. 2012; 28:544-549.
46. Kim et al., (2010) Modifications of small RNAs and their associated proteins. Cell, 143, 703-709.
47. Jayaprakash et al., (2011) Identification and remediation of biases in the activity of RNA ligases in small-RNA deep sequencing. Nucleic Acids Res., 39, e141.
48. Zhuang et al., (2012) Structural bias in T4 RNA ligase-mediated 3′-adapter ligation. Nucleic Acids Res., 40, e54.
49. Sorefan et al., (2012) Reducing ligation bias of small RNAs in libraries for next generation sequencing. Silence, 3, 4-15.
50. Kosaka et al., (2010) Circulating microRNA in body fluid: a new potential biomarker for cancer diagnosis and prognosis. Cancer Sci., 101, 2087-2092.
51. Wang et al., (2010) Circulating microRNA: a novel potential biomarker for early diagnosis of acute myocardial infarction in humans. Eur. Heart J., 31, 659-666.
52. Wang et al., (2010) Export of microRNAs and microRNA-protective protein by mammalian cells. Nucleic Acids Res., 38, 7248-7259.
53. Recchioni et al., (2013) Conventional and novel diagnostic biomarkers of acute myocardial infarction: a promising role for circulating microRNAs. Biomarkers, 18, 547-558.
54. Pritchard et al., (2012) Blood cell origin of circulating microRNAs: a cautionary note for cancer biomarker studies. Cancer Prev. Res. (Phila), 5, 492-497.
55. Kirschner et al., (2013) The impact of hemolysis on cell-free microRNA biomarkers. Front. Genet., 4, 94-107.
56. Kirschner et al., (2011) Haemolysis during sample preparation alters microRNA content of plasma. PLoS ONE, 6, e24145.
57. Hafner et al., (2012) Barcoded cDNA library preparation for small RNA profiling by next-generation sequencing. Methods, 58, 164-170.
58. Burgos et al., (2013) Identification of extracellular miRNA in human cerebrospinal fluid by next-generation sequencing. RNA, 19, 712-722.
59. Kivioja et al., (2012) Counting absolute numbers of molecules using unique molecular identifiers. Nat. Methods, 9, 72-74.
60. Shiroguchi et al., (2012) Digital RNA sequencing minimizes sequence-dependent bias and amplification noise with optimized single-molecule barcodes. Proc. Natl. Acad. Sci. U.S.A., 109, 1347-1352.
61. Casbon et al., (2011) A method for counting PCR template molecules with application to next-generation sequencing. Nucleic Acids Res., 39, e81.
62. Fu et al., (2011) Counting individual DNA molecules by the stochastic attachment of diverse labels. Proc. Natl. Acad. Sci. U.S.A., 108, 9026-9031.
63. Blondal et al., (2013) Assessing sample and miRNA profile quality in serum and plasma or other biofluids. Methods, 59, S1-S6.
64. McDonald et al., (2011) Analysis of circulating microRNA: preanalytical and analytical challenges. Clin. Chem., 57, 833-840.
65. Rasmussen et al., (2010) The miR-144/451 locus is required for erythroid homeostasis. J. Exp. Med., 207, 1351-1358.
66. Cheng et al., (2013) Plasma processing conditions substantially influence circulating microRNA biomarker levels. PLoS ONE, 8, e64795.
67. Aravin et al., (2006) A novel class of small RNAs bind to MILI protein in mouse testes. Nature, 442, 203-207.
68. Girard et al., (2006) A germline-specific class of smallRNAs binds mammalian Piwi proteins. Nature, 442, 199-202.
69. Grivna et al., (2006) A novel class of small RNAs in mouse spermatogenic cells. Genes Dev., 20, 1709-1714.
70. Watanabe et al., (2006) Identification and characterization of two novel classes of small RNAs in the mouse germline: retrotransposon-derived siRNAs in oocytes and germline small RNAs in testes. Genes Dev., 20, 1732-1743.
71. Nakamura et al., (2007) Laser capture microdissection for analysis of single cells. Methods Mol. Med., 132, 11-18.
72. Redmond et al., (2014) Laser capture microdissection of embryonic cells and preparation of RNA for microarray assays. Methods Mol. Biol., 1092, 43-60.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of preparing a cDNA library from RNA molecules in a biological sample, comprising the steps of:
providing a 3′ DNA adapter annealed to a unique DNA oligonucleotide, wherein the unique DNA oligonucleotide comprises a first portion that is complementary to the 3′ DNA adapter and a second portion;
ligating the 3′ DNA adapter to RNA molecules, wherein the ligating is performed under conditions that optimize the ligation reaction;
reverse transcribing the RNA molecules in the presence of at least one labeled nucleotide to produce labeled cDNA molecules, wherein the reverse transcribing is performed under conditions that optimize the reverse transcription;
circularizing the labeled cDNA molecules, wherein the circularizing is performed under conditions that optimize the circularization reaction;
optionally purifying or isolating the cDNA molecules;
linearizing and performing a first amplification of the cDNA molecules; and
purifying the amplification product,
thereby preparing a cDNA library.

2. The method of claim 1, further comprising performing a second amplification of the product from the first amplification to add platform-specific adapters.

3. The method of claim 1, wherein the unique DNA oligonucleotide further comprises a randomer.

4. The method of claim 1, wherein the RNA molecules are total plasma RNA molecules.

5. The method of claim 1, wherein the RNA molecules are small, circulating RNA molecules.

6. The method of claim 1, wherein the RNA molecules are microRNA molecules.

7. The method of claim 1, wherein the conditions that optimize the ligation reaction comprise carrying out the reaction in the presence of at least a 5:1 molar excess ratio of the 3′ DNA adapter:RNA molecules.

8. The method of claim 1, wherein the conditions that optimize the ligation reaction comprise carrying out the reaction for 6 hours at 30 C.

9. The method of claim 1, wherein the labeled nucleotide is a biotinylated labeled nucleotide.

10. The method of claim 1, wherein the reverse transcribing step is performed in the presence of two labeled nucleotides.

11. The method of claim 1, wherein the conditions that optimize the reverse transcription reaction comprise carrying out the reaction at a temperature that avoids denaturation of the 3′ DNA adapter and the unique DNA oligonucleotide.

12. The method of claim 1, further comprising removing the RNA molecules and precipitating the labeled cDNA molecules following the reverse transcribing step.

13. The method of claim 1, wherein the conditions that optimize the circularization reaction comprise carrying out the reaction in the presence of 25% of the recommended amount of enzyme.

14. The method of claim 13, wherein the enzyme is CircLigase I or CircLigase II single-stranded DNA ligase (Epicenter).

15. The method of claim 1, wherein the conditions that optimize the circularization reaction comprise carrying out the reaction in the presence of betaine.

16. The method of claim 1, further comprising purifying or isolating the circularized cDNA molecules.

17. The method of claim 16, wherein the purifying step is a gel purifying step.

18. The method of claim 16, wherein the purifying step further comprises a size selection step.

19. The method of claim 16, wherein the isolation is by streptavidin-labeled beads.

20. The method of claim 1, wherein the unique DNA oligonucleotide comprises at least one ideoxyU nucleotide and wherein the linearizing step is performed using UDG.

21. The method of claim 1, wherein the biological sample is selected from the group consisting of biofluids, biopsy tissue blocks, and cells isolated from laser capture microdissection.

22. The method of claim 1, wherein the biological sample comprises about 10 pg or less of total RNA.


SEQUENCE LISTING (52 sequences)

SEQ ID NO: 1 - DNA, 17 bp, Artificial Sequence
  synthetically generated oligonucleotide
ctgtaggcaccatcaat

SEQ ID NO: 2 - DNA, 19 bp, Artificial Sequence
  synthetically generated oligonucleotide
gatctacagtccgacgatc

SEQ ID NO: 3 - DNA, 17 bp, Artificial Sequence
  synthetically generated oligonucleotide
attgatggtgcctacag

SEQ ID NO: 4 - DNA, 57 bp, Artificial Sequence
  synthetically generated oligonucleotide
ccactacgcctccgctttcctctctatgggcagtcggtgatctacagtccgacgatc

SEQ ID NO: 5 - DNA, 47 bp, Artificial Sequence
  synthetically generated oligonucleotide
ccatctcatccctgcgtgtctccgactcagattgatggtgcctacag

SEQ ID NO: 6 - DNA, 17 bp, Artificial Sequence
  synthetically generated oligonucleotide
ctgtaggcaccatcaat

SEQ ID NO: 7 - DNA, 37 bp, Artificial Sequence
  DNA/RNA hybrid
tctacrargrurcrcrgrarcrgrarurcrurgrarc

SEQ ID NO: 8 - DNA, 37 bp, Artificial Sequence
  DNA/RNA hybrid
tctacrargrurcrcrgrarcrgrarurcrcrargru

SEQ ID NO: 9 - DNA, 50 bp, Artificial Sequence
  synthetically generated oligonucleotide
gnnnntgagatcgtcggactgtagaacgnnnccgattgatggtgcctaca

SEQ ID NO: 10 - DNA, 50 bp, Artificial Sequence
  synthetically generated oligonucleotide
gnnnnaacgatcgtcggactgtagaacgnnnccgattgatggtgcctaca

SEQ ID NO: 11 - DNA, 50 bp, Artificial Sequence
  synthetically generated oligonucleotide
gnnnnggcgatcgtcggactgtagaacgnnnccgattgatggtgcctaca

SEQ ID NO: 12 - DNA, 50 bp, Artificial Sequence
  synthetically generated oligonucleotide
gnnnngatgatcgtcggactgtagaacgnnnccgattgatggtgcctaca

SEQ ID NO: 13 - DNA, 50 bp, Artificial Sequence
  synthetically generated oligonucleotide
gnnnnagtgatcgtcggactgtagaacgnnnccgattgatggtgcctaca

SEQ ID NO: 14 - DNA, 50 bp, Artificial Sequence
  synthetically generated oligonucleotide
gnnnncccgatcgtcggactgtagaacgnnnccgattgatggtgcctaca

SEQ ID NO: 15 - DNA, 50 bp, Artificial Sequence
  synthetically generated oligonucleotide
gnnnntctgatcgtcggactgtagaacgnnnccgattgatggtgcctaca

SEQ ID NO: 16 - DNA, 50 bp, Artificial Sequence; identical to SEQ ID NO: 15
  synthetically generated oligonucleotide

SEQ ID NO: 17 - DNA, 50 bp, Artificial Sequence
  synthetically generated oligonucleotide
gnnnncttgatcgtcggactgtagaacgnnnccgattgatggtgcctaca

SEQ ID NO: 18 - DNA, 50 bp, Artificial Sequence
  synthetically generated oligonucleotide
gnnnntaagatcgtcggactgtagaacgnnnccgattgatggtgcctaca

SEQ ID NO: 19 - DNA, 50 bp, Artificial Sequence
  synthetically generated oligonucleotide
gnnnntcagatcgtcggactgtagaacgnnnccgattgatggtgcctaca

SEQ ID NO: 20 - DNA, 50 bp, Artificial Sequence
  synthetically generated oligonucleotide
gnnnntgtgatcgtcggactgtagaacgnnnccgattgatggtgcctaca

SEQ ID NO: 21 - DNA, 50 bp, Artificial Sequence
  synthetically generated oligonucleotide
gnnnnttggatcgtcggactgtagaacgnnnccgattgatggtgcctaca

SEQ ID NO: 22 - DNA, 50 bp, Artificial Sequence
  synthetically generated oligonucleotide
gnnnntatgatcgtcggactgtagaacgnnnccgattgatggtgcctaca

SEQ ID NO: 23 - DNA, 50 bp, Artificial Sequence
  synthetically generated oligonucleotide
gnnnnactgatcgtcggactgtagaacgnnnccgattgatggtgcctaca

SEQ ID NO: 24 - DNA, 50 bp, Artificial Sequence
  synthetically generated oligonucleotide
gnnnnacagatcgtcggactgtagaacgnnnccgattgatggtgcctaca

SEQ ID NO: 25 - DNA, 50 bp, Artificial Sequence
  synthetically generated oligonucleotide
gnnnnaaggatcgtcggactgtagaacgnnnccgattgatggtgcctaca

SEQ ID NO: 26 - DNA, 50 bp, Artificial Sequence; identical to SEQ ID NO: 20
  synthetically generated oligonucleotide

SEQ ID NO: 27 - DNA, 50 bp, Artificial Sequence
  synthetically generated oligonucleotide
gnnnngttgatcgtcggactgtagaacgnnnccgattgatggtgcctaca

SEQ ID NO: 28 - DNA, 50 bp, Artificial Sequence
  synthetically generated oligonucleotide
gnnnnattgatcgtcggactgtagaacgnnnccgattgatggtgcctaca

SEQ ID NO: 29 - DNA, 53 bp, Artificial Sequence
  synthetically generated oligonucleotide
gnnnnacatgtgatcgtcggactgtagaacgnnnccgattgatggtgcctaca

SEQ ID NO: 30 - DNA, 53 bp, Artificial Sequence
  synthetically generated oligonucleotide
gnnnnacgatagatcgtcggactgtagaacgnnnccgattgatggtgcctaca

SEQ ID NO: 31 - DNA, 53 bp, Artificial Sequence
  synthetically generated oligonucleotide
gnnnnagactcgatcgtcggactgtagaacgnnnccgattgatggtgcctaca

SEQ ID NO: 32 - DNA, 53 bp, Artificial Sequence
  synthetically generated oligonucleotide
gnnnnatcgcagatcgtcggactgtagaacgnnnccgattgatggtgcctaca

SEQ ID NO: 33 - DNA, 53 bp, Artificial Sequence
  synthetically generated oligonucleotide
gnnnncagagtgatcgtcggactgtagaacgnnnccgattgatggtgcctaca

SEQ ID NO: 34 - DNA, 53 bp, Artificial Sequence
  synthetically generated oligonucleotide
gnnnncatctcgatcgtcggactgtagaacgnnnccgattgatggtgcctaca

SEQ ID NO: 35 - DNA, 53 bp, Artificial Sequence
  synthetically generated oligonucleotide
gnnnngatacagatcgtcggactgtagaacgnnnccgattgatggtgcctaca

SEQ ID NO: 36 - DNA, 53 bp, Artificial Sequence
  synthetically generated oligonucleotide
gnnnngtagctgatcgtcggactgtagaacgnnnccgattgatggtgcctaca

SEQ ID NO: 37 - DNA, 53 bp, Artificial Sequence
  synthetically generated oligonucleotide
gnnnntctgctgatcgtcggactgtagaacgnnnccgattgatggtgcctaca

SEQ ID NO: 38 - DNA, 53 bp, Artificial Sequence
  synthetically generated oligonucleotide
gnnnntgctcagatcgtcggactgtagaacgnnnccgattgatggtgcctaca

SEQ ID NO: 39 - DNA, 53 bp, Artificial Sequence
  synthetically generated oligonucleotide
gnnnnatctcggatcgtcggactgtagaacgnnnccgattgatggtgcctaca

SEQ ID NO: 40 - DNA, 53 bp, Artificial Sequence
  synthetically generated oligonucleotide
gnnnngtctatgatcgtcggactgtagaacgnnnccgattgatggtgcctaca

SEQ ID NO: 41 - DNA, 53 bp, Artificial Sequence
  synthetically generated oligonucleotide
gnnnntcaatcgatcgtcggactgtagaacgnnnccgattgatggtgcctaca

SEQ ID NO: 42 - DNA, 53 bp, Artificial Sequence
  synthetically generated oligonucleotide
gnnnncgaatggatcgtcggactgtagaacgnnnccgattgatggtgcctaca

SEQ ID NO: 43 - DNA, 55 bp, Artificial Sequence
  synthetically generated oligonucleotide
gnnnnnnaagaacgatcgtcggactgtagaacgnnnccgattgatggtgcctaca

SEQ ID NO: 44 - DNA, 17 bp, Artificial Sequence
  synthetically generated oligonucleotide
attgatggtgcctacag

SEQ ID NO: 45 - DNA, 19 bp, Artificial Sequence
  synthetically generated oligonucleotide
gatctacagtccgacgatc

SEQ ID NO: 46 - DNA, 47 bp, Artificial Sequence; identical to SEQ ID NO: 5
  synthetically generated oligonucleotide

SEQ ID NO: 47 - DNA, 57 bp, Artificial Sequence; identical to SEQ ID NO: 4
  synthetically generated oligonucleotide

SEQ ID NO: 48 - DNA, 17 bp, Artificial Sequence
  synthetically generated oligonucleotide
attgatggtgcctacag

SEQ ID NO: 49 - DNA, 19 bp, Artificial Sequence
  synthetically generated oligonucleotide
gttctacagtccgacgatc

SEQ ID NO: 50 - DNA, 38 bp, Artificial Sequence
  synthetically generated oligonucleotide
acagcagaagacggcatacgaattgatggtgcctacag

SEQ ID NO: 51 - DNA, 48 bp, Artificial Sequence
  synthetically generated oligonucleotide
aatgatacggcgaccaccgacaggttcagagttctacagtccgacgat

SEQ ID NO: 52 - DNA, 17 bp, Artificial Sequence
  synthetically generated oligonucleotide
ctgtaggcaccatcaat